(19) United States
(12) Patent Application Publication
(10) Pub. No.: US 2009/0074775 A1
(43) Pub. Date: Mar. 19, 2009

(54) Use Of Anti-AB Antibody To Treat Traumatic Brain Injury

(76) Inventors: David Michael Holtzman, St. Louis, MO (US); David Lozoff Brody, St. Louis, MO (US)

Correspondence Address: PATRICK W. RASCHE (15060);ARMSTRONG TEASDALE, LLP, ONE METROPOLITAN SQUARE, SUITE 2600, SAINT LOUIS, MO, 63102-2740, US

(21) Appl. No.: 11/722,635

(22) PCT Filed: Dec. 21, 2005
(86) PCT No.: PCT/US2005/046208

§ 371 Date: Jun. 3, 2008

Related U.S. Application Data
(60) Provisional application No. 60/639,524, filed on Dec. 22, 2004.

(51) Int. Cl. A61K 39/395; A61P 25/00

(52) U.S. Cl. 424/139.1; 424/172.1

(57) ABSTRACT

A method of effectively treating traumatic brain injury is described. The method comprises administering an effective amount of an anti-Aβ antibody to a living mammalian biosystem such as to a human. An antibody useful in such treating includes an antibody that therapeutically attenuates the toxic effects of the Aβ peptide in a living mammal in relation to traumatic brain injury.

This application claims the benefit of U.S. Ser. No. 60/639,524 filed Dec. 22, 2004 which is incorporated herein in its entirety by reference.

### FIELD OF THE DISCOVERY

This discovery relates generally to a method effectively treating living patients with traumatic brain injury (hereinafter referred to as “TBI”). In particular this discovery relates to the use of an antibody to therapeutically attenuate at least one symptom or sign of TBI.

### BACKGROUND OF THE DISCOVERY

TBI is a major cause of death and neurological disability in humans. TBI includes those brain injuries occurring in motor vehicle accidents, after falls, caused by assault and in sports when force is applied to the head sufficiently to produce injury to the structure of the brain. Such injury can include bruising, tearing and swelling of brain tissue. It can include intracranial bleeding, such as subdural, epidural, subarachnoid, intraparenchymal and intraventricular hemorrhage. Brain tissue can be injured such as due to shearing of axons, even when little to no bleeding occurs.

Traumatic brain injury (TBI) is a major cause of death and severe disability, with an estimated incidence of 1.5 million new cases per year in the United States, unfortunately resulting in 50,000 fatalities. A total of 5.3 million Americans, approximately 2% of the U.S. population, currently live with disabilities resulting from TBI. Because many of the victims are young, total costs are extremely high and are estimated at about $56 billion per year (Thurman et al., 1999). Despite extensive research over many years at several large clinical trials, there are currently no effective treatments for TBI other than meticulous supportive care (Narayan et al., 2002).

Amyloid-beta (Aβ) is a 38-43 amino acid peptide derived from an amyloid precursor protein (APP). Undesired build-up of amyloid-β (or Aβ) has been studied primarily in the context of patients with Alzheimer's Disease and Down syndrome. However, a detailed autopsy series found diffuse Aβ plaques in 46 of 152 (30%) cases of fatal TBI, including in patients as young as 10 years old without Down syndrome or autosomal dominant familial AD (Roberts et al., 1991; Roberts et al., 1994). Some of the deposits were found to contain amyloid by Congo red staining in another study that confirmed these results (Huber et al., 1993). Aβ plaques were not confined to the sites of direct injury, but instead spread throughout the cortex and hippocampus (Graham et al., 1995). They were seen preferentially in areas of diffuse axonal injury, as localized by APP or neurofilament-stained swollen axons (Smith et al., 2003), suggesting that release of Aβ from injured axons may be part of the underlying mechanism. This pathology was present in patients who survived for as little as 4 hours after injury and were under 60 years old, indicating that a deposition can occur quickly (Roberts et al., 1994). However, the post-traumatic pathology included few deposits of Aβ in the form of true amyloid (by thioflavine-S or congo-red staining), and neurofibrillary tangles were not consistently reported. Aβ pathology has been reported in human patients who survived TBI but had portions of their brains surgically resected to control elevated intracranial pressure (Ikonomovic et. al. 2004). The importance of this Aβ pathology had not been previously established.

Increases in Aβ concentrations or acceleration of Aβ plaque formation have been observed in animal models of TBI. (Smith et al., 1998, Uryu et. al. 2002, Hartman et al., 2002, Chen et al., 2004). However, there have been no previous experimental interventions aimed at reducing Aβ levels or attenuating its toxicity in the setting of TBI. Thus, it was previously unknown whether these increases in Aβ concentrations or acceleration of Aβ plaque formation were pathogenically important.

### BRIEF DESCRIPTION OF THE INVENTION

In an aspect, a method of effectively treating at least one clinically detectable symptom or sign of traumatic brain injury comprises administering an effective amount of an anti-Aβ antibody to a living human patient. In an aspect, an antibody useful in such treatment includes an antibody that therapeutically attenuates the toxic effects of the Aβ peptide in a living mammal.

In an aspect, a medicinal composition useful to treat at least one clinically detectable symptom or sign of traumatic brain injury comprises a medicinally effective amount of an anti-Aβ antibody adapted for administration to a living human patient. In an aspect, an antibody useful in such treatment includes an antibody that therapeutically attenuates the toxic effects of the Aβ peptide in a living mammal. In an aspect, the medicinal composition is effectively administered to a living patient.

In an aspect, a medicinal kit comprising a container containing a functional therapeutic medicinal composition of a medicinally effective amount of an anti-Aβ antibody adapted for administration to a living human patient and any medical devices to be used for said administration. In an aspect, an antibody useful in such treatment includes an antibody that therapeutically attenuates the toxic effects of the Aβ peptide in a living mammal.

### BRIEF DESCRIPTION OF THE DRAWINGS

Drawings 1A, 1B, 1C, 1D, 2, 3A, 3B, 4, 5, 6, and 7 provide data illustrating this discovery.

FIGS. 1A-1D are graphs showing behavioral performance of mice during Morris water maze testing. The effects on performance of anti-Aβ antibody treatment in the setting of experimental TBI and the effects of the experimental TBI itself are demonstrated.

FIG. 2 is a graph depicting the results of the water maze probe trial, a specific test of spatial memory.

FIG. 3A is a line drawing depicting examples of various search strategies used by mice during the performance of the water maze test.

FIG. 3B is a graph demonstrating the effects of anti-Aβ antibody treatment and experimental TBI on search strategy use during water maze testing.

FIG. 4 is a graph showing that there is no significant effect of anti-Aβ antibody treatment on water maze performance in young PDAPP mice not subjected to TBI.

FIG. 5 is a graph providing quantitative histological analysis of the effects of experimental TBI and anti-Aβ antibody treatment on cortical and hippocampal volumes.

FIG. 6 is a graph providing quantitative histological analysis of the effects of experimental TBI and anti-Aβ antibody treatment on hippocampal CA3 neuronal cell counts.

FIG. 7 is a graph providing quantitative histological analysis of the effects of experimental TBI and anti-Aβ antibody treatment on newly generated neurons (BrdU-NeuN double labeled cells) in the dentate gyrus.

### DETAILED DESCRIPTION OF THE INVENTION

Applicants have discovered a method of effectively treating traumatic brain injury which comprises effectively administering a pharmacologically effective amount of anti-Aβ antibody to a living patient having TBI. The present invention encompasses the discovery that anti-Aβ antibodies provide a treatment for patients suffering from TBI as they beneficially affect the cognitive impairment and neuronal damage associated with TBI. Thus, the invention provides evidence that signs and symptoms of TBI may be due, at least in part, to the deleterious effects of Aβ. In an aspect at least one preclinical or clinical symptom or sign is presented by that patient. In an aspect, an antibody useful in such treating includes an antibody that therapeutically attenuates the toxic effects of the Aβ peptide in a living mammal. In an aspect antibodies useful in such treating include those which bind an epitope within positions 13-28 of Aβ.

In an aspect, an anti-Aβ antibody is admixed with at least one suitable compatible adjuvant or excipient resulting in a therapeutic medicinal composition which is capably and effectively administered (given) to a living patient, such as to a human afflicted with TBI Typically this is an aqueous composition of high purity.

As used herein, the terms “treating” or “treatment” include prevention, attenuation, reversal, or improvement in at least one symptom or sign of traumatic brain injury.

As used herein the term “therapeutically attenuate” includes inducing a change or having a beneficial positive effect resulting therefrom.

One definition of TBI is provided in the Individuals with Disabilities Education Act which defines traumatic brain injury as “an acquired injury to the brain caused by an external physical force, resulting in total or partial functional disability or psychosocial impairment, or both, that adversely affects a child's educational performance. The term applies to both open and closed head injuries resulting in impairments in one or more areas, such as cognition; language; memory; attention; reasoning; abstract thinking; judgment; problem-solving; sensory, perceptual, and motor abilities; psycho-social behavior; physical functions; information processing; and speech. [34 Code of Federal Regulations §300.7(c)(12)] TBI occurs in people of all ages, including infants and children, young adults, adults and elderly. A similar definition applies to people of all ages, with the modification that work-related, cognitive, behavioral, emotional and social performance impairments can be involved in addition to adverse effects on educational performance.

Signs and symptoms of TBI include impaired cognitive function, altered behavior, emotional dysregulation, seizures, headaches, impaired nervous system structure or function, and an increased risk of development of Alzheimer's disease. Impaired cognitive function includes but is not limited to difficulties with memory, attention, concentration, abstract thought, creativity, executive function, planning, and organization. Altered behavior includes but is not limited to physical or verbal aggression, impulsivity, decreased inhibition, apathy, decreased initiation, changes in personality, abuse of alcohol, tobacco or drugs, and other addiction-related behaviors. Emotional dysregulation includes but is not limited to depression, anxiety, mania, irritability, and emotional incontinence. Seizures include but are not limited to generalized tonic-clonic seizures, complex partial seizures, and non-epileptic, psychogenic seizures. Headaches include but are not limited to common migraine, classic migraine, complex or atypical migraine, cluster headache and tension headache. Impaired nervous system structure or function includes but is not limited to hydrocephalus, parkinsonism, sleep disorders, psychosis, impairment of balance and coordination. This includes motor impairments such as monoparesis, hemiparesis, tetraparesis, ataxia, ballismus and tremor. This also includes sensory loss or dysfunction including olfactory, tactile, gustatory, visual and auditory sensation. Furthermore, this includes autonomic nervous system impairments such as bowel and bladder dysfunction, sexual dysfunction, blood pressure and temperature dysregulation. Finally, this includes hormonal impairments attributable to dysfunction of the hypothalamus and pituitary gland such as deficiencies and dysregulation of growth hormone, thyroid stimulating hormone, lutenizing hormone, follicle stimulating hormone, gonadotropin releasing hormone, prolactin, and numerous other hormones and modulators. Increased risk of development of Alzheimer's disease includes that risk that is elevated over the expected risk given the patients age, family history, genetic status and other known risk factors.

The diagnosis of TBI is made based on clinical history and physical exam findings. A clinical history leading to the diagnosis of TBI includes but is not limited to one obtained from the patient or witness indicating that physical force was applied to the head directly or indirectly sufficient to produce impairment of the function of the brain. Physical exam findings leading to the diagnosis of TBI include but are not limited to injuries to the skin and bones indicating that physical force has been applied to the head and evidence of impaired function of the brain. Radiological studies such as X-rays, CT scans and MRI scans are used to support a diagnosis of TBI, but are neither necessary nor sufficient to make the diagnosis. There are no blood, urine, CSF or other laboratory tests that are either necessary or sufficient to make the diagnosis of TBI.

Aβ peptides are those derived from a region in the carboxy terminus of a larger protein called amyloid precursor protein (APP). The gene encoding APP is located on chromosome 21. There are many forms of Aβ that may have toxic effects: Aβ peptides are typically 38-43 amino acids long, though they can have truncations and modifications changing their overall size. They can be found in soluble and insoluble compartments, in monomeric, oligomeric and aggregated forms, intracellularly or extracellularly, and may be complexed with other proteins or molecules. The adverse or toxic effects of Aβ may be attributable to any or all of the above noted forms, as well as to others not described specifically.

Anti-Aβ antibodies useful herein include all antibodies that therapeutically attenuate the adverse or toxic effects of Aβ. These include but are not limited to those antibodies disclosed in PCT/US02/26321 (WO 03/015617A2) published Feb. 27, 2003, the contents of which are incorporated herein in its entirety by reference. Useful antibodies include but are not limited to those that specifically bind to an epitope within the region defined by amino acids 13 to 28 in Aβ peptides. Anti-Aβ antibodies useful herein include also antibodies that attenuate the adverse or toxic effects of Aβ and bind to other regions of Aβ and to other forms of Aβ. Other regions of Aβ include but are not limited to the C-terminal, the N-terminal, and other central domains. Other forms of Aβ include but are not limited to truncated, modified, soluble, insoluble, intracellular, extracellular, monomeric Aβ, oligomeric Aβ, fibrillar, aggregated Aβ or Aβ complexed with other proteins or molecules.

Anti-Aβ antibodies useful herein include but are not limited to those antibodies and fragments thereof, wherein the variable regions have sequences comprising the one or more of the CDRs from mouse antibody 266 and specific human framework sequences (SEQ ID NO:7 through SEQ ID NO:10). Especially useful are humanized antibodies and fragments thereof, wherein the light chain has a sequence corresponding to the sequence shown in SEQ ID NO:11 and the heavy chain has a sequence corresponding to the sequence shown in SEQ ID NO:12.

Patents WO 01/62801 and WO 04/071408, the contents of which are incorporated herein in its entirety by reference, describe the preparation of examples of useful anti-Aβ antibodies. These include a monoclonal antibody, designated clone 266 (m266), which was reportedly raised against, and has reportedly been shown to bind specifically to, a peptide comprising amino acids 13-28 of the Aβ peptide.

In an aspect, antibodies useful herein include those antibodies which have been isolated, characterized, purified, are functional and have been recovered (obtained) for use in a functional therapeutic composition which is administered to a living patient having TBI.

“Monoclonal antibody” refers to an antibody that is derived from a single copy or clone, including e.g., any eukaryotic, prokaryotic, or phage clone. “Monoclonal antibody” is not limited to antibodies produced through hybridoma technology. Monoclonal antibodies can be produced using e.g., hybridoma techniques well known in the art, as well as recombinant technologies, phage display technologies, synthetic technologies or combinations of such technologies and other technologies readily known in the art. Furthermore, the monoclonal antibody may be labeled with a detectable label, immobilized on a solid phase and/or conjugated with a heterologous compound (e.g., an enzyme or toxin) according to methods known in the art.

Further by “antibody” is meant a functional monoclonal antibody, or an immunologically effective fragment thereof; such as an Fab, Fab′, or F(ab′)2 fragment thereof. In some contexts herein, fragments will be mentioned specifically for emphasis; nevertheless, it will be understood that regardless of whether fragments are specified, the term “antibody” includes such fragments as well as single-chain forms. As long as the protein retains the ability specifically to bind its intended target, and in this case, to bind Aβ peptide in blood and/or the CNS, it is included within the term “antibody.” Also included within the definition “antibody” for example are single chain forms, generally designated Fv, regions, of antibodies with this specificity. Preferably, but not necessarily, the antibodies useful in the discovery are produced recombinantly, as manipulation of the typically murine or other non-human antibodies with the appropriate specificity is required in order to convert them to humanized form. Antibodies may or may not be glycosylated, though glycosylated antibodies are preferred. Antibodies are properly cross-linked via disulfide bonds, as is known.

The basic antibody structural unit of an antibody useful herein comprises a tetramer. Each tetramer is composed of two identical pairs of polypeptide chains, each pair having one “light’ (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Anti-Aβ antibodies useful herein include those which are isolated, characterized, purified, functional and have been recovered (obtained) from a process for their preparation and thus available for use herein in a useful form in a therapeutically and medicinally sufficient amount.

Light chains are classified as gamma, mu, alpha, and lambda. Heavy chains are classified as gamma, mu, alpha, delta, or epsilon, and define the antibody's isotype as IgO, IgM, IgA, IgD and IgE, respectively. Within light and heavy chains, the variable and constant regions are joined by a “J” region of about 12 or more amino acids, with the heavy chain also including a “D” region of about 10 more amino acids.

The variable regions of each light/heavy chain pair form the antibody binding site. Thus, an intact antibody has two binding sites. The chains exhibit the same general structure of relatively conserved framework regions (FR) joined by three hypervariable regions, also called complementarily determining regions (hereinafter referred to as “CDRs.”) The CDRs from the two chains are aligned by the framework regions, enabling binding to a specific epitope. From N-terminal to C-terminal, both light and heavy chains comprise the domains FR1, CDR1, FR2, CDR2, FR3, CDR3 and FR4 respectively. The assignment of amino acids to each domain is in accordance with known conventions [See, Kabat “Sequences of Proteins of Immunological Interest” National Institutes of Health, Bethesda, Md., 1987 and 1991; Chothia, et al, J. Mol. Bio. (1987) 196:901-917; Chothia, et al., Nature (1989) 342:878-883].

In an aspect, monoclonal anti-Aβ antibodies are generated with appropriate specificity by standard techniques of immunization of mammals, forming hybridomas from the antibody-producing cells of said mammals or otherwise immortalizing them, and culturing the hybridomas or immortalized cells to assess them for the appropriate specificity. In the present case such antibodies could be generated by immunizing a human, rabbit, rat or mouse, for example, with a peptide representing an epitope encompassing the 13-28 region of the Aβ peptide or an appropriate subregion thereof. Materials for recombinant manipulation can be obtained by retrieving the nucleotide sequences encoding the desired antibody from the hybridoma or other cell that produces it. These nucleotide sequences can then be manipulated and isolated, characterized, purified and, recovered to provide them in humanized form, for use herein if desired.

As used here “humanized antibody” includes an anti-Aβ antibody that is composed partially or fully of amino acid sequences derived from a human antibody germline by altering the sequence of an antibody having non-human complementarity determining regions (“CDR”). The simplest such alteration may consist simply of substituting the constant region of a human antibody for the murine constant region, thus resulting in a human/murine chimera which may have sufficiently low immunogenicity to be acceptable for pharmaceutical use. Preferably, however, the variable region of the antibody and even the CDR is also humanized by techniques that are by now well known in the art. The framework regions of the variable regions are substituted by the corresponding human framework regions leaving the non-human CDR substantially intact, or even replacing the CDR with sequences derived from a human genome. CDRs may also be randomly mutated such that binding activity and affinity for Aβ is maintained or enhanced in the context of fully human germline framework regions or framework regions that are substantially human. Substantially human frameworks have at least 90%, 95%, or 99% sequence identity with a known human framework sequence. Fully useful human antibodies are produced in genetically modified mice whose immune systems have been altered to correspond to human immune systems. As mentioned above, it is sufficient for use in the methods of this discovery, to employ an immunologically specific fragment of the antibody, including fragments representing single chain forms.

Further, as used herein the term “humanized antibody” refers to an anti-Aβ antibody comprising a human framework, at least one CDR from a nonhuman antibody, and in which any constant region present is substantially identical to a human immunoglobulin constant region, i.e., at least about 85-90%, preferably at least 95% identical. Hence, all parts of a humanized antibody, except possibly the CDRs, are substantially identical to corresponding pairs of one or more native human immunoglobulin sequences.

If desired, the design of humanized immunoglobulins may be carried out as follows. When an amino acid falls under the following category, the framework amino acid of a human immunoglobulin to be used (acceptor immunoglobulin) is replaced by a framework amino acid from a CDR-providing nonhuman immunoglobulin (donor immunoglobulin): (a) the amino acid in the human framework region of the acceptor immunoglobulin is unusual for human immunoglobulin at that position, whereas the corresponding amino acid in the donor immunoglobulin is typical for human immunoglobulin at that position; (b) the position of the amino acid is immediately adjacent to one of the CDRs; or (c) any side chain atom of a framework amino acid is within about 5-6 angstroms (center-to-center) of any atom of a CDR amino acid in a three dimensional immunoglobulin model (Queen, et al., op. cit., and Co, ct al, Proc. Natl. Acad. Sci. USA (1991) 88:2869]. When each of the amino acid in the human framework region of the acceptor immunoglobulin and a corresponding amino acid in the donor immunoglobulin is unusual for human immunoglobulin at that position, such an amino acid is replaced by an amino acid typical for human immunoglobulin at that position.

A preferred humanized antibody useful herein in this discovery, is a humanized form of mouse antibody 266. The CDRs of humanized 266 have the following respective amino acid sequences:

light chain CDR1:

(SEQ ID NO:1)

1 5 10
Arg Ser Ser Gln Ser Leu Ile Tyr Ser Asp Gly Asn
15
Ala Tyr Leu His
light chain CDR2:

(SEQ ID NO:2)

1 5
Lys Val Ser Asn Arg Phe Ser
light chain CDR3:

(SEQ ID NO:3)

1 5
Ser Gln Ser Thr His Val Pro Trp Thr
heavy chain CDR1:

(SEQ ID NO:4)

1 5
Arg Tyr Ser Met Ser
heavy chain CDR2:

(SEQ ID NO:5)

1 5 10
Gln Ile Asn Ser Val Gly Asn Ser Thr Tyr Tyr Pro
15
Asp Thr Val Lys Gly
and,
heavy chain CDR3:

(SEQ ID NO:6)

1
Gly Asp Tyr

A preferred light chain variable region of a humanized antibody of the present discovery has the following amino acid sequence, in which the framework originated from human germline Vk segments DPK18 and J segment Jk1, with several amino acid substitutions to the consensus amino acids in the same human V subgroup to reduce potential immunogenicity:

(SEQ ID NO:7)

```
1 5 10
Asp Xaa Val Met Thr Gln Xaa Pro Leu Ser Leu Pro
                  15 20
Val Xaa Xaa Gly Gln Pro Ala Ser Ile Ser Cys Arg
  25 30 35
Ser Ser Gln Ser Leu Xaa Tyr Ser Asp Gly Asn Ala
                          40 45
Tyr Leu His Trp Phe Leu Gln Lys Pro Gly Gln Ser
          50 55 60
Pro Xaa Leu Leu Ile Tyr Lys Val Ser Asn Arg Phe
                                  65 70
Ser Gly Val Pro Asp Arg Phe Ser Gly Ser Gly Ser
                  75 80
Gly Thr Asp Phe Thr Leu Lys Ile Ser Arg Val Glu
  85 90 95
Ala Glu Asp Xaa Gly Val Tyr Tyr Cys Ser Gln Ser
                        100 105
Thr His Val Pro Trp Thr Phe Gly Xaa Gly Thr Xaa
        110
Xaa Glu Ile Lys Arg
```

wherein:

Xaa at position 2 is Val or Ile;

Xaa at position 7 is Ser or Thr;

Xaa at position 14 is Thr or Ser;

Xaa at position 15 is Leu or Pro;

Xaa at position 30 is Ile or Val;

Xaa at position 50 is Arg, Gln, or Lys;

Xaa at position 88 is Val or Leu;

Xaa at position 105 is Gln or Gly

Xaa at position 108 is Lys or Arg; and

Xaa at position 109 is Val or Leu.

A preferred heavy chain variable region of a humanized antibody of the present discovery has the following amino acid sequence, in which the framework originated from human germline VH segments DP53 and J segment JH4, with several amino acid substitutions to the consensus amino acids in the same human subgroup to reduce potential immunogenicity:

(SEQ ID NO:8)

```
1 5 10
Xaa Val Gln Leu Val Glu Xaa Gly Gly Gly Leu Val
                  15 20
Gln Pro Gly Gly Ser Leu Arg Leu Ser Cys Ala Ala
  25 30 35
Ser Gly Phe Thr Phe Ser Arg Tyr Ser Met Ser Trp
                          40 45
Val Arg Gln Ala Pro Gly Lys Gly Leu Xaa Leu Val
          50 55 60
Ala Gln Ile Asn Ser Val Gly Asn Ser Thr Tyr Tyr
                                  65 70
Pro Asp Xaa Val Lys Gly Arg Phe Thr Ile Ser Arg
                  75 80
Asp Asn Xaa Xaa Asn Thr Leu Tyr Leu Gln Met Asn
  85 90 95
Ser Leu Arg Ala Xaa Asp Thr Ala Val Tyr Tyr Cys
                        100 105
Ala Ser Gly Asp Tyr Trp Gly Gln Gly Thr Xaa Val
        110
Thr Val Ser Ser
```

wherein:

Xaa at position 1 is Glu or Gln;

Xaa at position 7 is Ser or Leu;

Xaa at position 46 is Glu, Val, or Ser;

Xaa at position 63 is Thr or Ser;

Xaa at position 75 is Ala, Ser, Val, or Thr;

Xaa at position 76 is Lys or Arg;

Xaa at position 89 is Glu or Asp; and

Xaa at position 107 is Leu or Thr.

A particularly preferred light chain variable region of a humanized antibody of the present discovery has the following amino acid sequence, in which the framework originated from human germline Vk segments DPK18 and J segment JkI, with several amino acid substitutions to the consensus amino acids in the same human V subgroup to reduce potential immunogenicity:

(SEQ ID NO:9)

```
1 5 10
Asp Val Val Met Thr Gln Ser Pro Leu Ser Leu Pro
                  15 20
Val Thr Leu Gly Gln Pro Ala Ser Ile Ser Cys Arg
  25 30 35
Ser Ser Gln Ser Leu Ile Tyr Ser Asp Gly Asn Ala
                          40 45
Tyr Leu His Trp Phe Leu Gln Lys Pro Gly Gln Ser
          50 55 60
Pro Arg Leu Leu Ile Tyr Lys Val Ser Asn Arg Phe
                                  65 70
Ser Gly Val Pro Asp Arg Phe Ser Gly Ser Gly Ser
                  75 80
Gly Thr Asp Phe Thr Leu Lys Ile Ser Arg Val Glu
  85 90 95
Ala Glu Asp Val Gly Val Tyr Tyr Cys Ser Gln Ser
                        100 105
Thr His Val Pro Trp Thr Phe Gly Gln Gly Thr Lys
        110
Val Glu Ile Lys Arg
```

A particularly preferred heavy chain variable region of a humanized antibody of the present discovery has the following amino acid sequence, in which the framework originated from human germline VH segments DP53 and J segment JH4:

(SEQ ID NO:10)

```
1 5 10
Glu Val Gln Leu Val Glu Ser Gly Gly Gly Leu Val
                  15 20
Gln Pro Gly Gly Ser Leu Arg Leu Ser Cys Ala Ala
  25 30 35
Ser Gly Phe Thr Phe Ser Arg Tyr Ser Met Ser Trp
                          40 45
Val Arg Gln Ala Pro Gly Lys Gly Leu Glu Leu Val
          50 55 60
Ala Gln Ile Asn Ser Val Gly Asn Ser Thr Tyr Tyr
                                  65 70
Pro Asp Thr Val Lys Gly Arg Phe Thr Ile Ser Arg
                  75 80
Asp Asn Ala Lys Asn Thr Leu Tyr Leu Gln Met Asn
  85 90 95
Ser Leu Arg Ala Glu Asp Thr Ala Val Tyr Tyr Cys
                        100 105
Ala Ser Gly Asp Tyr Trp Gly Gln Gly Thr Leu Val
        110
Thr Val Ser Ser
```

A preferred light chain for a humanized antibody of the present discovery has the amino acid sequence:

(SEQ ID NO:11)

Asp Val Val Met Thr Gln Ser Pro Leu Ser Leu Pro
Val Thr Leu Gly Gln Pro Ala Ser Ile Ser Cys Arg
Ser Ser Gln Ser Leu Ile Tyr Ser Asp Gly Asn Ala
Tyr Leu His Trp Phe Leu Gln Lys Pro Gly Gln Ser
Pro Arg Leu Leu Ile Tyr Lys Val Ser Asn Arg Phe
Ser Gly Val Pro Asp Arg Phe Ser Gly Ser Gly Ser
Gly Thr Asp Phe Thr Leu Lys Ile Ser Arg Val Glu
Ala Glu Asp Val Gly Val Tyr Tyr Cys Ser Gln Ser
Thr His Val Pro Trp Thr Phe Gly Gln Gly Thr Lys
Val Glu Ile Lys Arg Thr Val Ala Ala Pro Ser Val
Phe Ile Phe Pro Pro Ser Asp Glu Gln Leu Lys Ser
Gly Thr Ala Ser Val Val Cys Leu Leu Asn Asn Phe
Tyr Pro Arg Glu Ala Lys Val Gln Trp Lys Val Asp
Asn Ala Leu Gln Ser Gly Asn Ser Gln Glu Ser Val
Thr Glu Gln Asp Ser Lys Asp Ser Thr Tyr Ser Leu
Ser Ser Thr Leu Thr Leu Ser Lys Ala Asp Tyr Glu
Lys His Lys Val Tyr Ala Cys Glu Val Thr His Gln
Gly Leu Ser Ser Pro Val Thr Lys Ser Phe Asn Arg
Gly Glu Cys.

A preferred heavy chain for a humanized antibody of the present discovery has the amino acid sequence:

(SEQ ID NO:12)

Glu Val Gln Leu Val Glu Ser Gly Gly Gly Leu Val
Gln Pro Gly Gly Ser Leu Arg Leu Ser Cys Ala Ala
Ser Gly Phe Thr Phe Ser Arg Tyr Ser Met Ser Trp
Val Ary Gln Ala Pro Gly Lys Gly Leu Glu Leu Val
Ala Gln Ile Asn Ser Val Gly Asn Ser Thr Tyr Tyr
Pro Asp Thr Val Lys Gly Arg Phe Thr Ile Ser Arg
Asp Asn Ala Lys Asn Thr Leu Tyr Leu Gln Met Asn
Ser Leu Arg Ala Glu Asp Thr Ala Val Tyr Tyr Cys
Ala Ser Gly Asp Tyr Trp Gly Gln Gly Thr Leu Val
Thr Val Ser Ser Ala Ser Thr Lys Gly Pro Ser Val
Phe Pro Leu Ala Pro Ser Ser Lys Ser Thr Ser Gly
Gly Thr Ala Ala Leu Gly Cys Leu Val Lys Asp Tyr
Phe Pro Glu Pro Val Thr Val Ser Trp Asn Ser Gly
Ala Leu Thr Ser Gly Val His Thr Phe Pro Ala Val
Leu Gln Ser Ser Gly Leu Tyr Ser Leu Ser Ser Val
Val Thr Val Pro Ser Ser Ser Leu Gly Thr Gln Thr
Tyr Ile Cys Asn Val Asn His Lys Pro Ser Asn Thr
Lys Val Asp Lys Lys Val Glu Pro Lys Ser Cys Asp
Lys Thr His Thr Cys Pro Pro Cys Pro Ala Pro Glu
Leu Leu Gly Gly Pro Ser Val Phe Leu Phe Pro Pro
Lys Pro Lys Asp Thr Leu Met Ile Ser Arg Thr Pro
Glu Val Thr Cys Val Val Val Asp Val Ser His Glu
Asp Pro Glu Val Lys Phe Asn Trp Tyr Val Asp Gly
Val Glu Val His Asn Ala Lys Thr Lys Pro Arg Glu
Glu Gln Tyr Asn Ser Thr Tyr Arg Val Val Ser Val
Leu Thr Val Leu His Gln Asp Trp Leu Asn Gly Lys
Glu Tyr Lys Cys Lys Val Ser Asn Lys Ala Leu Pro
Ala Pro Ile Glu Lys Thr Ile Ser Lys Ala Lys Gly
Gln Pro Ary Glu Pro Gln Val Tyr Thr Leu Pro Pro
Ser Arg Asp Gln Leu Thr Lys Asn Gln Val Ser Leu
Thr Cys Leu Val Lys Gly Phe Tyr Pro Ser Asp Ile
Ala Val Glu Trp Glu Ser Asn Gly Gln Pro Glu Aso
Asn Tyr Lys Thr Thr Pro Pro Val Leu Asp Ser Asp
Gly Ser Phe Phe Leu Tyr Ser Lys Leu Thr Val Asp
Lys Ser Arg Trp Gln Gln Gly Asn Val Phe Ser Cys
Ser Val Met His Glu Ala Leu His Asn His Tyr Thr
Gln Lys Ser Leu Ser Leu Ser Pro Gly Lys

Other sequences are possible for the light and heavy chains for useful humanized antibodies of the present discovery and for humanized 266. The immunoglobulins can have two pairs of light chain/heavy chain complexes, at least one chain comprising one or more mouse complementarity determining regions functionally joined to human framework region segments.

Starting at position 56 of the heavy chain variable region, both m266 and humanized 266 contain the sequence Asn-Ser-Thr. This sequence is an example of the Asn-X-Ser/Thr signal for N-linked glycosylation, wherein the Asn is the site of attachment of N-linked glycosyl chains. Both m266 and humanized 266 are extensively glycosylated at this site. Another preferred antibody for use in the present discovery is an analog of 266, in which an N-glycosylation site within CDR2 of the heavy chain is engineering so as not to be glycosylated. The heavy chain CDR2 of deglycosylated humanized 266 has the following amino acid sequences:

heavy chain CDR2:

(SEQ ID NO:13)

1 5 10
Gln Ile Asn Ser Val Gly Xaa Xaa Xaa Tyr Tyr Pro
15
Asp Thr Val Lys Gly

Wherein:

Xaa at position 7 is any amino acid, provided that if Xaa at position 8 is neither Asp nor Pro and Xaa at position 9 is Ser or Thr, then Xaa at position 7 is not Asn;

Xaa at position 8 is any amino acid, provided that if Xaa at position 7 is Asn and Xaa at position 9 is Ser or Thr, then Xaa at position 8 is Asp or Pro; and

Xaa at position 9 is any amino acid, provided that if Xaa at position 7 is Asn and Xaa at position 8 is neither Asp nor Pro, then Xaa at position 9 is neither Ser nor Thr;

By “any amino acid” is meant any naturally-occurring amino acid. Preferred naturally-occurring amino acids are Ala, Cys, Asp, Glu, Phe, Gly, His, Ile, Lys, Leu, Met, Asn, Pro, Gln, Arg, Ser, Thr, Val, Trp, and Tyr.

A preferred deglycosylated humanized antibody useful herein is a humanized form of m266, wherein the deglycosylated heavy chain CDR2 is SEQ ID NO:13, wherein: Xaa at position 7 of SEQ ID NO: 13 is selected from the group consisting of Ala, Cys, Asp, Glu, Phe, Gly, His, Ile, Lys, Leu, Met, Asn, Pro, Gln, Arg, Ser, Thr, Val, Trp, and Tyr, provided that if Xaa at position 8 is neither Asp nor Pro and Xaa at position 9 is Ser or Thr, then Xaa at position 7 is not Asn;

Xaa at position 8 of SEQ ID NO:13 is selected from the group consisting of Ala, Cys, Asp, Glu, Phe; Gly, His, Ile, Lys, Leu, Met, Asn, Pro, Gln, Arg, Ser, Thr, Val, Trp, and Tyr, provided that if Xaa at position 7 is Asn and Xaa at position 9 is Ser or Thr, then Xaa at position 8 is Asp or Pro; and

Xaa at position 9 of SEQ ID NO: 13 is selected from the group consisting of Ala, Cys, Asp, Glu, Phe, Gly, His, Ile, Lys, Leu, Met, Asn, Pro, Gin, Arg, Ser, Thr, Val, Trp, and Tyr, provided that if Xaa at position 7 is Asn and Xaa at position 8 is neither Asp nor Pro, then Xaa at position 9 is neither Ser nor Thr.

A preferred heavy chain variable region of a deglycosylated humanized antibody of the present discovery has the following amino acid sequence, in which the framework originated from human germline VH segment DP53 and J segment JH4, with several amino acid substitutions to the consensus amino acids in the same human subgroup to reduce potential immunogenicity and wherein the N-glycosylation site in heavy chain CDR2 is modified so that it cannot be N-glycosylated:

(SEQ ID NO:14)

```
1 5 10
Xaa Val Gln Leu Val Glu Xaa Gly Gly Gly Leu Val
                  15 20
Gln Pro Gly Gly Ser Leu Arg Leu Ser Cys Ala Ala
  25 30 35
Ser Gly Phe Thr Phe Ser Arg Tyr Ser Met Ser Trp
                          40 45
Val Arg Gln Ala Pro Gly Lys Gly Leu Xaa Leu Val
          50 55 60
Ala Gln Ile Asn Ser Val Gly Xaa Xaa Xaa Tyr Tyr
                                  65 70
Pro Asp Xaa Val Lys Gly Arg Phe Thr Ile Ser Arg
                  75 80
Asp Asn Xaa Xaa Asn Thr Leu Tyr Leu Gln Met Asn
  85 90 95
Ser Leu Arg Ala Xaa Asp Thr Ala Val Tyr Tyr Cys
                        100 105
Ala Ser Gly Asp Tyr Trp Gly Gln Gly Thr Xaa Val
        110
Thr Val Ser Ser
```

wherein:

Xaa at position 1 is Glu or Gln;

Xaa at position 7 is Ser or Leu;

Xaa at position 46 is Glu, Val, Asp, or Ser;

Xaa at position 56 is any amino acid, provided that if Xaa at position 57 is neither Asp nor Pro and Xaa at position 59 is Ser or Thr, then Xaa at position 56 is not Asn;

Xaa at position 57 is any amino acid, provided that if Xaa at position 56 is Asn and Xaa at position 58 is Ser or Thr, then Xaa at position 57 is Asp or Pro; and

Xaa at position 58 is any amino acid, provided that if Xaa at position 56 is Asn and Xaa at position 57 is neither Asp nor Pro, then Xaa at position 58 is neither Ser nor Thr

Xaa at position 63 is Thr or Ser;

Xaa at position 75 is Ala, Ser, Val, or Thr;

Xaa at position 76 is Lys or Arg;

Xaa at position 89 is Glu or Asp; and

Xaa at position 107 is Leu or Thr.

A particularly preferred heavy chain variable region of a deglycosylated humanized antibody of the present discovery has the following amino acid sequence, in which the framework originated from human germline Vii segment DP53 and J segment JH4 and wherein the N-glycosylation site in heavy chain CDR2 is modified so that it cannot be N-glycosylated:

(SEQ ID NO:15)

```
1 5 10
Glu Val Gln Leu Val Glu Ser Gly Gly Gly Leu Val
                  15 20
Gln Pro Gly Gly Ser Leu Arg Leu Ser Cys Ala Ala
  25 30 35
Ser Gly Phe Thr Phe Ser Arg Tyr Ser Met Ser Trp
                          40 45
Val Arg Gln Ala Pro Gly Lys Gly Leu Glu Leu Val
          50 55 60
Ala Gln Ile Asn Ser Val Gly Xaa Xaa Xaa Tyr Tyr
                                  65 70
Pro Asp Thr Val Lys Gly Arg Phe Thr Ile Ser Arg
                  75 80
Asp Asn Ala Lys Asn Thr Leu Tyr Leu Gln Met Asn
  85 90 95
Ser Leu Arg Ala Gln Asp Thr Ala Val Tyr Tyr Cys
                        100 105
Ala Ser Gly Asp Tyr Trp Gly Gln Gly Thr Leu Val
        110
Thr Val Ser Ser
```

wherein:

Xaa at position 56 is any amino acid, provided that if Xaa at position 57 is neither Asp nor Pro and Xaa at position 59 is Ser or Thr, then Xaa at position 56 is not Asn;

Xaa at position 57 is any amino acid, provided that if Xaa at position 56 is Asn and Xaa at position 58 is Ser or Thr, then Xaa at position 57 is Asp or Pro; and

Xaa at position 58 is any amino acid, provided that if Xaa at position 56 is Asn and Xaa at position 57 is neither Asp nor Pro, then Xaa at position 58 is neither Ser nor Thr.

A preferred heavy chain for a deglycosylated humanized, antibody of the present discovery, wherein the N-glycosylation site in heavy chain. CDR2 is modified so that it cannot be N-glycosylated, has the amino acid sequence:

(SEQ ID NO:16)

```
1 5 10
Glu Val Gln Leu Val Glu Ser Gly Gly Gly Leu Val
                  15 20
Gln Pro Gly Gly Ser Leu Arg Leu Ser Cys Ala Ala
  25 30 35
Ser Gly Phe Thr Phe Ser Arg Tyr Ser Met Ser Trp
                          40 45
Val Arg Gln Ala Pro Gly Lys Gly Leu Glu Leu Val
          50 55 60
Ala Gln Ile Asn Ser Val Gly Xaa Xaa Xaa Tyr Tyr
                                  65 70
Pro Asp Thr Val Lys Gly Arg Phe Thr Ile Ser Arg
                  75 80
Asp Asn Ala Lys Asn Thr Leu Tyr Leu Gln Met Asn
  85 90 95
Ser Leu Arg Ala Glu Asp Thr Ala Val Tyr Tyr Cys
                        100 105
Ala Ser Gly Asp Tyr Trp Gly Gln Gly Thr Leu Val
        110 115 120
Thr Val Ser Ser Ala Ser Thr Lys Gly Pro Ser Val
                                125 130
Phe Pro Leu Ala Pro Ser Ser Lys Ser Thr Ser Gly
                135 140
Gly Thr Ala Ala Leu Gly Cys Leu Val Lys Asp Tyr
145 150 155
Phe Pro Glu Pro Val Thr Val Ser Trp Asn Ser Gly
                        160 165
Ala Leu Thr Ser Gly Val His Thr Phe Pro Ala Val
        170 175 180
Leu Gln Ser Ser Gly Leu Tyr Ser Leu Ser Ser Val
                                185 190
Val Thr Val Pro Ser Ser Ser Leu Gly Thr Gln Thr
                195 200
Tyr Ile Cys Asn Val Asn His Lys Pro Ser Asn Thr
205 210 215
Lys Val Asp Lys Lys Val Glu Pro Lys Ser Cys Asp
                        220 225
Lys Thr His Thr Cys Pro Pro Cys Pro Ala Pro Glu
        230 235 240
Leu Leu Gly Gly Pro Ser Val Phe Leu Phe Pro Pro
                                245 250
Lys Pro Lys Asp Thr Leu Met Ile Ser Arg Thr Pro
                255 260
Glu Val Thr Cys Val Val Val Asp Val Ser His Glu
265 270 275
Asp Pro Glu Val Lys Phe Asn Trp Tyr Val Asp Gly
                        280 285
Val Glu Val His Asn Ala Lys Thr Lys Pro Arg Glu
        290 295 300
Glu Gln Tyr Asn Ser Thr Tyr Arg Val Val Ser Val
                                305 310
Leu Thr Val Leu His Gln Asp Trp Leu Asn Gly Lys
                315 320
Glu Tyr Lys Cys Lys Val Ser Asn Lys Ala Leu Pro
325 330 335
Ala Pro Ile Glu Lys Thr Ile Ser Lys Ala Lys Gly
                        340 345
Gln Pro Arg Glu Pro Gln Val Tyr Thr Leu Pro Pro
        350 355 360
Ser Arg Asp Glu Leu Thr Lys Asn Gln Val Ser Leu
                                365 370
Thr Cys Leu Val Lys Gly Phe Tyr Pro Ser Asp Ile
                375 380
Ala Val Glu Trp Glu Ser Asn Gly Gln Pro Glu Asn
385 390 395
Asn Tyr Lys Thr Thr Pro Pro Val Leu Asp Ser Asp
                        400 405
Gly Ser Phe Phe Leu Tyr Ser Lys Leu Thr Val Asp
        410 415 420
Lys Ser Arg Trp Gln Gln Gly Asn Val Phe Ser Cys
                                425 430
Ser Val Met His Glu Ala Leu His Asn His Tyr Thr
                435 440
Gln Lys Ser Leu Ser Leu Ser Pro Gly Lys
```

Xaa at position 56 is any amino acid, provided that if Xaa at position 57 is neither Asp nor Pro and Xaa at position 59 is Ser or Thr, then Xaa at position 56 is not Asn

Xaa at position 57 is any amino acid, provided that if Xaa at position 56 is Asn and Xaa at position 58 is Ser or Thr, then Xaa at position 57 is Asp or Pro; and

Xaa at position 58 is any amino acid, provided that if Xaa at position 56 is Asn and Xaa at position 57 is neither Asp nor Pro, then Xaa at position 58 is neither Ser nor Thr.

Preferred deglycosylated 266 antibodies having the heavy variable region according to SEQ ID NO: 14, SEQ ID NO: 15, and SEQ ID NO: 16 are those wherein:

Xaa at position 56 is selected from the group consisting of Ala, Gly, His, Asn, Gln, Ser, and Thr, provided that if Xaa at position 58 is Ser or Thr, then Xaa at position 56 is not Asn

Xaa at position 57 is selected from the group consisting of Ala, Gly, His, Asn, Gln, Ser, and Thr and

Xaa at position 58 is selected from the group consisting of Ala, Gly, His, Asn, Gln, Ser, and Thr, provided that if Xaa at position 56 is Asn, then Xaa at position 58 is neither Ser nor Thr.

Preferred sequences for CDR2 (positions 56, 57, and 58) of the heavy chain SEQ ID NO:14, SEQ ID NO:15, and SEQ ID NO:16 include those in which only a single amino acid is changed, those in which only two amino acids are changed, or all three are changed. It is preferred to replace Asn at position 56. It is preferred to replace Thr at position 58 with an amino acid other than Ser. It is preferred to not destroy the N-glycosylation site in the CDR2 of the 266 heavy chain by replacing Ser at position 57 with Pro or Asp. Conservative substitutions at one, two, or all three positions are preferred. The most preferred species are those in which Asn at position 56 is replaced with Ser or Thr. Particularly preferred antibodies are those in which Ser or Thr is at position 56, Ser is at position 57, and Thr is at position 58 of SEQ ID NO:14, SEQ ID NO:15, or SEQ ID NO:16.

Especially preferred deglycosylated species are antibodies comprising a light chain of SEQ ID NO: 11 and a heavy chain of SEQ ID NO:16, wherein in SEQ ID NO: 16, Xaa at position 56 is Ser, Xaa at position 57 is Ser, and Xaa at position 58 is Thr (“N56S”), or wherein in SEQ ID NO: 16, Xaa at position 56 is Thr, Xaa at position 57 is Ser, and Xaa at position 58 is Thr (“NS6T”).

Production of the antibodies useful in the discovery typically involves recombinant techniques, as is described in PCT/US01/06191 now EP 1481 992 A3 published Dec. 8, 2004 which is incorporated herein by reference in its entirety.

In an aspect, the antibodies in a pharmacologically effective amount preferred in pharmaceutical grade, including immunologically reactive fragments, are administered to a subject such as to a living patient to be treated for traumatic brain injury. Administration is performed using standard effective techniques, include peripherally (i.e. not by administration into the central nervous system) or locally to the central nervous system. Peripheral administration includes but is not limited to intravenous, intraperitoneal, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. It may be particularly useful to alter the solubility characteristics of the antibodies useful in this discovery, making them more lipophilic, for example, by encapsulating them in liposomes or by blocking polar groups.

Effective peripheral systemic delivery by intravenous or intraperitoneal or subcutaneous injection is a preferred method of administration to a living patient. Suitable vehicles for such injections are straightforward. In addition, however, administration may also be effected through the mucosal membranes by means of nasal aerosols or suppositories. Suitable formulations for such modes of administration are well known and typically include surfactants that facilitate cross-membrane transfer. Such surfactants are often derived from steroids or are cationic lipids, such as N-[1-(2,3-dioleoyl)propyl]-N,N,N-trimethyl ammonium chloride (DOTMA) or various compounds such as cholesterol hemisuccinate, phosphatidyl glycerols and the like.

The concentration of humanized antibody in formulations to be administered is an effective amount and ranges from as low as about 0.1% by weight to as much as about 15 or about 20% by weight and will be selected primarily based on fluid volumes, viscosities, and so forth, in accordance with the particular mode of administration selected if desired. A typical composition for injection to a living patient could be made up to contain 1 mL sterile buffered water of phosphate buffered saline and about 1-1000 mg of the humanized antibody of the present discovery. The formulation could be sterile filtered after making the formulation, or otherwise made microbiologically acceptable. A typical composition for intravenous infusion could have volumes between 1-250 mL of fluid, such as sterile Ringer's solution, and 1-100 mg per ml, or more in anti-Aβ antibody concentration. Therapeutic agents of the discovery can be frozen or lyophilized for storage and reconstituted in a suitable sterile carrier prior to use. Lyophilization and reconstitution can lead to varying degrees of antibody activity loss (e.g. with conventional immune globulins, IgM antibodies tend to have greater activity loss than IgG antibodies). Dosages administered are effective dosages and may have to be adjusted to compensate. The pH of the formulations generally pharmaceutical grade quality, will be selected to balance antibody stability (chemical and physical) and comfort to the patient when administered. Generally, a pH between 4 and 8 is tolerated. Doses will vary from individual to individual based on size, weight, and other physiobiological characteristics of the individual receiving the successful administration.

As used herein, the term “effective amount” means an amount of a substance such as a compound that leads to measurable and beneficial effects for the patient administered the substance, i.e., significant efficacy. The effective amount or dose of compound administered according to this discovery will be determined by the circumstances surrounding the case, including the compound administered, the route of administration, the status of the TBI being treated and similar patient and administration situation considerations among other considerations. In an aspect a typical dose contains from about 0.01 mg/kg to about 100 mg/kg of an anti-Aβ antibody described herein. Doses can range from about 0.05 mg/kg to about 50 mg/kg, more preferably from about 0.1 mg/kg to about 25 mg/kg. The frequency of dosing may be daily or once, twice, three times or more per week or per month, as needed as to effectively treat the condition of TBI.

The timing of administration of the treatment relative to the injury itself and duration of treatment will be determined by the circumstances surrounding the case. Treatment could begin immediately, such as at the site of the injury as administered by emergency medical personnel. Treatment could begin in a hospital itself, or at a later time after discharge from the hospital. Duration of treatment could range from a single dose administered on a one-time basis to a life-long course of therapeutic treatments.

Although the foregoing methods appear the most convenient and most appropriate and effective for administration of proteins such as humanized antibodies, by suitable adaptation, other effective techniques for administration, such as intraventricular administration, transdermal administration and oral administration may be employed provided proper formulation is utilized herein.

In addition, it may be desirable to employ controlled release formulations using biodegradable films and matrices, or osmotic mini-pumps, or delivery systems based on dextran beads, alginate, or collagen.

Typical dosage levels can be determined and optimized using standard clinical techniques and will be dependent on the mode of administration.

The examples below employ a functional murine monoclonal antibody designated “m266” which was originally prepared by immunization with a peptide comprised of residues 13-28 of human Aβ peptide. The antibody was confirmed to immunoreact with this peptide, but had previously been reported to not react with the peptide containing only residues 17-28 of human Aβ peptide, or at any other epitopes within the Aβ peptide. The preparation of this antibody is described in WO 01/62801, incorporated herein by reference in its entirety.

### EXAMPLES

Exemplary embodiments are described in the following examples. It is intended that the specification, together with the examples, be considered exemplary only.

Overview: Efficacy of Anti-Aβ Antibody Treatment in Experimental TBI Performed in Transgenic Mice Producing Human Aβ

Transgenic mice that express a mutant human amyloid precursor protein (PDAPP mice) and produce human Aβ are subjected to experimental TBI. 500 micrograms of an anti-Aβ antibody (m266) is given intraperitoneally 12 hours before and then weekly after TBI. Spatial learning is assessed using the Morris water maze. BrdU is injected daily for 7 days following TBI to label dividing cells. Newly generated neurons are counted using confocal imaging of BrdU, NeuN colocalization.

Systemic administration of this antibody to PDAPP mice improves cognitive performance following TBI. The PDAPP mice perform significantly better in the Morris water maze 18-21 days after TBI than a placebo group, and are comparable to a 3rd group of PDAPP mice that did not receive TBI. The number of newly generated neurons in the dentate gyrus is increased and CA3 cell loss is reduced in the antibody-treated group compared to placebo treated mice. There are no differences between the two groups in the overall size of the lesions in hippocampus and cortex, and minimal to no deposition of Aβ or amyloid formation in any of the PDAPP mice at age 5-6 months.

It was discovered that an effective anti-Aβ antibody treatment dramatically improves cognitive function following test induced TBI with relatively subtle effects on the histologically defined lesion. This dissociation suggests that changes in soluble Aβ handling and metabolism after TBI contribute to cognitive impairment. Thus, anti-Aβ antibody treatment appears to neutralize toxic effects of Aβ that worsen cognitive performance after acute TBI.

Experimental Design

First, mice are tested in the Morris water maze before injury. Then, the PDAPP mice are assigned to 3 groups: TBI alone, TBI with anti-Aβ antibody pretreatment, or sham TBI. Wild type mice are assigned to TBI, sham TBI, or naive groups. Mice are divided according to their baseline water maze performance so that the groups all have balanced numbers of mice performing well, average or poorly. Identical matched vials of anti-Aβ antibody solution and bovine serum albumin solution are prepared and labeled only with the coded group labels.

Mice receive effective intraperitoneal injections of either bovine serum albumin solution or 500 μg of m266 12-16 hours before traumatic brain injury. They are then subjected to a left parasagittal, controlled cortical impact TBI of moderate severity. The mice are then treated weekly with antibody or placebo in a blinded fashion for an additional 4 weeks. Half of the mice in each group receive daily i.p. injections of 50 mg/kg BrdU for 7 days starting on the day of injury. The other half receives identical injections of saline only. Thirteen days after TBI, all surviving mice are retested in the Morris water maze. Testing is performed in a different room, the platform is placed in a different location, and different spatial cues are used. Mice are sacrificed under pentobarbital anesthesia (65 mg/kg i.p.) 1 month after TBI, at 5-6 months of age.

Experimental Traumatic Brain Injury

Mice undergo a single, moderate left lateral controlled cortical intentional impact with craniotomy, as described previously (Dixon et al., 1991; Smith et al., 1995; Murai et al., 1998). Mice are anesthetized i.p. with 65 mg/kg pentobarbital. 10 minutes later, ointment to protect vision was applied to their eyes, and they are placed in a stereotactic frame on a warming pad. The top of the skull is exposed and a 5 mm craniotomy is performed over the left parietotemporal cortex using a hand trephine. Care is taken not to penetrate the dura during this procedure. 45 minutes after anesthesia, animals are subjected to controlled cortical impact (“CCI”) in which a 3 mm flat metal tip impounder is driven by a pneumatic cylinder at a velocity of 5 m/s to a depth of 1 mm into the cortex. Sham-treated animals are anesthetized, have a craniotomy, and are placed in the CCI device but do not undergo CCI TBI. Mice are removed from the CCI device, the stereotactic frame detached, and a plastic skull cap placed under the skin covering the craniotomy site. The skin is then closed with interrupted 4-0 silk sutures and mice are allowed to recover on a warming pad. They are returned to and placed in their home cages when fully ambulatory, about 1.5 hours after induction of anesthesia.

Morris Water Maze Testing

This test comprises a water pool with a hidden escape platform wherein the subject (e.g. mouse) has to learn how to find the platform with local or visual clues (Morris et al., 1982). In this behavioral test, mice are placed in a pool of opacified water containing a hidden platform just below the surface of the water. They escape from the maze (are removed from the pool) when they find the hidden platform. Distal visual cues are arrayed around the room, and in general mice are able to learn the location of the hidden platform based on these cues. Performance in this test is believed to reflect spatial learning and memory (Morris, 1984; Crawley, 2000; D'Hooge and De Deyn, 2001) and it is sensitive to disruption by TBI (Smith et al., 1995; Murai et al., 1998; Nakagawa et al., 1999; Uryu et al., 2002).

Because PDAPP mice perform poorly in Morris water maze testing even without traumatic brain injury (Smith et al., 1998; Chen et al., 2000), the protocol is modified to facilitate learning. All tests are run at night; as 3-5 month old PDAPP mice appear to have more pronounced circadian rhythm in body temperature and activity than WT mice (Huitron-Resendiz et al., 2002). The platform was made ˜50% larger (16 cm diameter vs. the typical 11 cm) which can improve learning (Crawley, 2000). Each mouse is allowed eight trials per day instead of the usual four trials per day. After arriving on the platform, the mouse is allowed to rest 30 seconds instead of 10. Prominent spatial cues are used including geometric shapes, posters of natural scenes, and a radio receiving and providing all talk radio to the mice.

For visible platform (cued) testing, a 1.09 m diameter pool is filled with room temperature (26° C.-28° C.) water. For 3 consecutive days, each mouse is placed in the pool in each of 4 starting locations arrayed around the pool. A clearly visible 16 cm diameter plastic platform is placed in one location throughout the 3 days. An automated tracking system (SMART, San Diego Instruments or Polytrack, San Diego Instruments, 7758 Arjons Drive, San Diego, Calif. 92126-4391, U.S.A.) records and analyzes the mouse swim paths. Each trial lasts a maximum of 60 seconds; and at the end of each trial, the mouse is placed on the platform or allowed to stay on the platform for 30 seconds. Each mouse is returned to its cage between trials, observed for signs of hypothermia, and warmed with a lamp if necessary. Mice that do not swim to the platform consistently in under 15 seconds by the 3rd day are excluded from further testing. In one experiment, no mice were excluded prior to TBI and eight mice were excluded after TBI; one or two animals from each group except for the naïve WT group were among the eight.

For hidden platform (place) testing, the platform is placed one cm under the surface of the water made opaque by a suspension of white, non-toxic tempera paint. It is placed in a different location from that used in visible platform testing. Each mouse is released from one of 4 locations and had 60 seconds to search for the hidden platform. At the end of each trial, the mouse is placed on the platform or allowed to stay on the platform for 30 seconds. Prominent spatial cues are arrayed around the room. The human investigator is also a powerful spatial cue and always sits in the same location during each trial after releasing the mouse. Eight trials per day for 5 consecutive days are performed with the location of the platform kept constant.

For the probe trial, performed the day after the completion of hidden platform testing, the platform is removed, and each mouse is placed in the pool once for about 30 seconds, starting from the same starting location that was used first in hidden platform testing. The time each mouse spends swimming in the quadrant where the platform had been is recorded.

Histology and Immunofluorescence

Mice are humanely anesthetized with pentobarbital and perfused with heparinized saline according to accepted standards. Brains are carefully removed, fixed in paraformaldehyde, and equilibrated in 30% sucrose (Holtzman et al., 2000). Every sixth 50 μm frozen section is mounted on glass slides (Fisher Scientific Intl, Inc., 1 Liberty Lane, Hampton, N.H. 03842, U.S.A., Superfrost Plus). Aβ immunofluorescence labeling is performed using 3D6, a monoclonal antibody that recognizes amino acids 1-5 of Aβ (38) conjugated to Alexa-568 (Molecular Probes Inc., 29851 Willow Creek Road, Eugene, Oreg. 97402, U.S.A.). The antibody is applied at a 1:500 dilution for 3 hours at room temperature in 1% powdered milk and then sections are washed thoroughly with 0.125% triton-X100. Amyloid staining is performed using thioflavine-S at 0.025% in 50% ethanol for 5 minutes at room temperature and then sections are washed serially in 50% alcohol, water, and saline. Aβ is visualized using a rhodamine filter cube (Omega XF38), and amyloid using a UV cube (Choma UVI-A) on an epifluorescence microscope (Nikon, 1300 Walt Whitman Road, Melville N.Y. 11747 USA).

The area covered by the hippocampus is traced in a separate set of bis-benzamide stained sections using Stereo Investigator design based stereology software (MicroBrightField, 185 Allen Brook Lane, Suite 201, Willistin, Utah 05495 USA). Volumes are estimated by summing the areas and multiplying by the spacing between sections (300 μm). Dorsal cortical volumes from the same set of sections are also estimated, with the inferior border of the dorsal cortex defined by the bottom margin of the dorsal third ventricle.

Neurons in the inferior blade of CA3 are counted in bis-benzamide stained sections using stereological methods as described (Hartman et al., 2002). Briefly, the inferior blade of CA3 is traced in each section using Stereo Investigator. Then, rounded neuronal nuclei are counted with a 100× lens using the optical fractionator technique throughout the entire rostral to caudal extent of the hippocampus. Each counting region is 15 μm thick with a 5 μm guard zone. The size of the counting frames ranged from 30×30 μm to 60×60 μm as needed to keep the Gunderson coefficients of error (CE) under 0.1 for each animal. It is necessary to adjust the counting frame size because of extensive tissue loss in some animals. An estimate of total cells per CA3 region is obtained multiplying the number of cells counted (typically ˜200) by the total volume of the inferior blade of CA3 and then dividing by the actual assessed volume.

BrdU, NeuN double immunofluorescence labeling is performed in a third set free-floating Sections (Kuhn et al., 1996; Arvidsson et al., 2001). Sections are washed in PBS with 0.25% triton X 100. Double stranded DNA is denatured to expose BrdU incorporated into DNA by treatment in 1 M HCl for 1 hour at 65° C. Lipofuscin autofluorescence is removed with 10 mM cupric sulfate in 50 mM ammonium acetate for 15 minutes at room temperature. Nonspecific binding is blocked with 5% normal goat serum and 5% normal donkey serum. The primary antibodies used are mouse anti-NeuN at 1:100 (Chemicon International, Inc., 2880 Single Oak Drive, Temecula, Calif. 92950 USA MAB377) and rat anti-BrdU at 1:200 (Harlan, P.O. Box 29176, Indianapolis, Ind. 46229-0176, USA OBT0030). These are incubated with 2% goat and 2% donkey serum for 36 hours at 4° C. The secondary antibodies used are goat anti-mouse IgG conjugated to Alexa 488 at 1:100 (Molecular Probes, inc., 29851 Willow Creek Road, Eugene, Oreg. 97402, USA A1 1029) and donkey anti-rat IgG conjugated to Cy3 at 1:200 (The Jackson Laboratory, 600 Main Street, Barr Harbor, Me. 04609, USA, 712-165-153). These are applied for 2 hours at room temperature. Sections are mounted with Prolong antifade reagent (Molecular Probes). Images are obtained on a Zeiss confocal microscope (Carl Zeiss Inc., One Zeiss Drive, Thornwood, N.Y. 10594 USA) using a 40× water immersion objective. For each animal, four 50 micron thick sections are chosen. These were spaced every 600 μm from the anterior to the posterior extent of the dentate gyrus. For each section, both dentate gyri are imaged using 3-8 partially overlapping stacks of images. A double labeled cell is defined as a volume of BrdU, NeuN co-localization at least 4 μm across in each of the 3 dimensions.

Administration of Anti-Aβ Antibody Improves Cognitive Performance Following Experimental TBI in APP Transgenic Mice.

PDAPP and WT mice that were subjected to TBI had a significant impairment in performance on the Morris water maze relative to pre-injury performance and relative to sham-treated mice that did not receive TBI (p<10⁻⁶ Repeated measures ANOVA). This indicates that the model of experimental TBI, controlled cortical impact, causes reproducible behavioral dysfunction, a common sign of TBI in human patients, and is therefore useful as a preclinical model of human TBI.

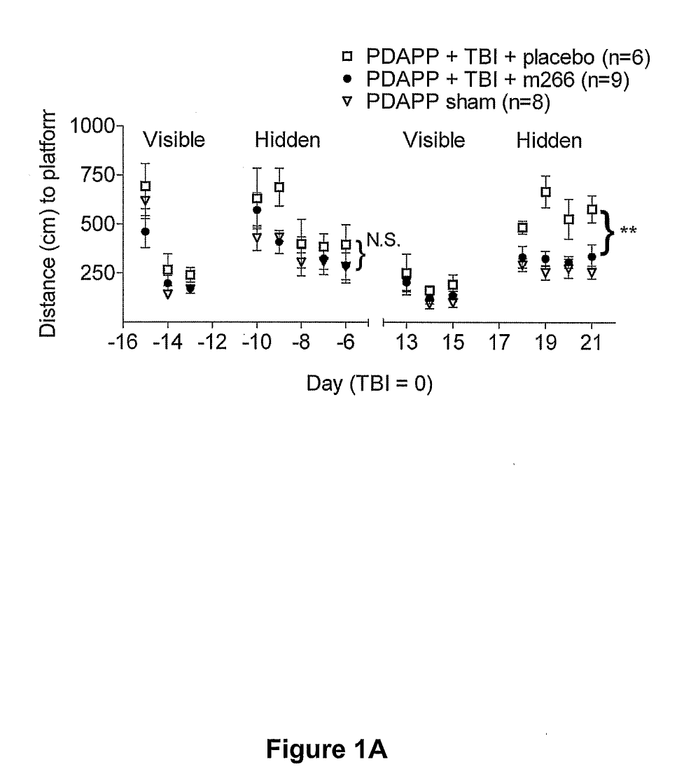

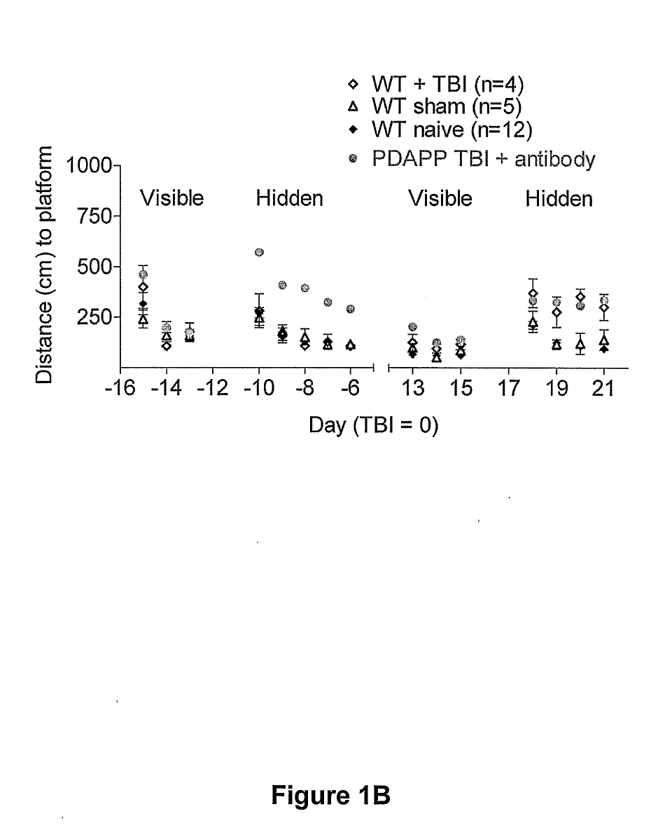

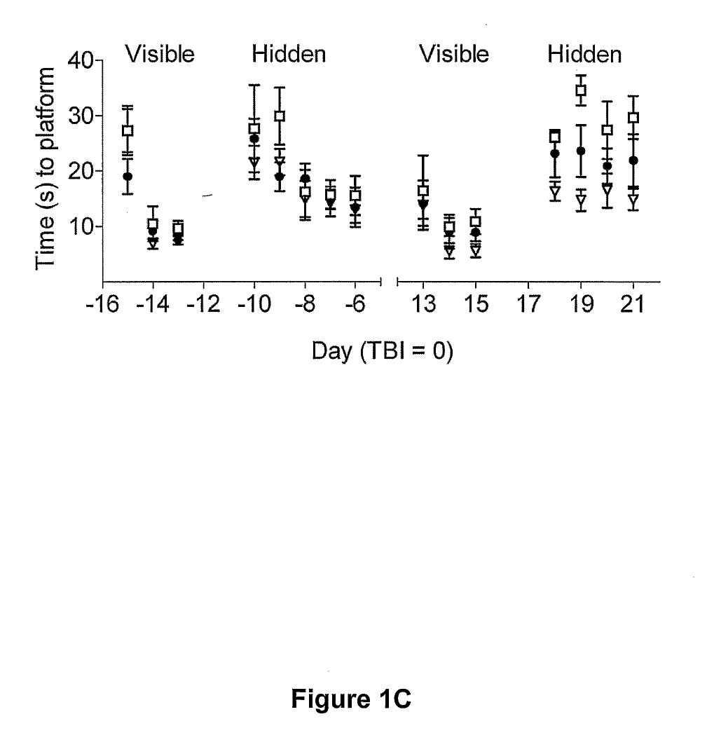

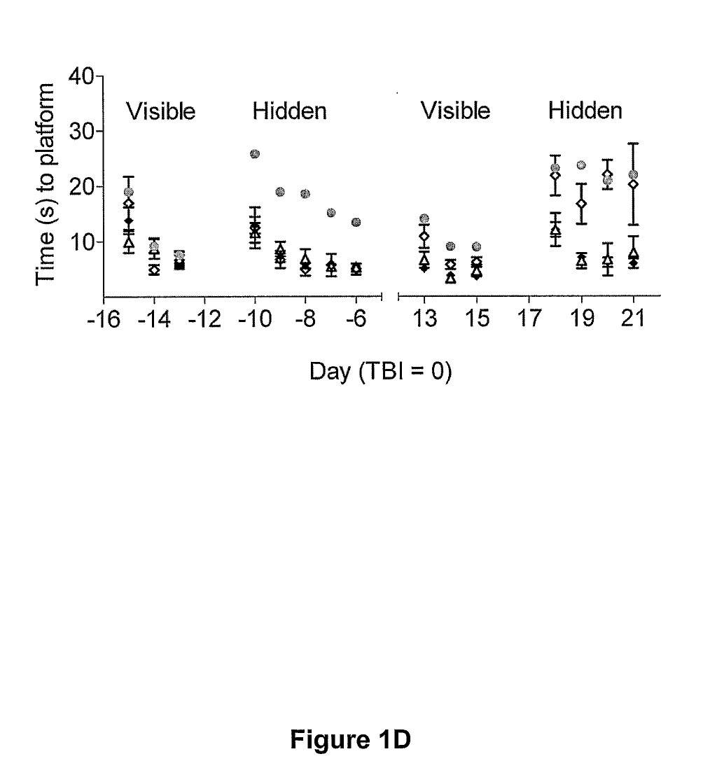

Hidden platform performance during Morris water maze testing was improved following anti-Aβ antibody treatment in PDAPP mice subjected to experimental TBI. FIG. 1A shows the distance to reach the platform in PDAPP mice. 4-5 month old PDAPP+/−mice are tested before and after moderate controlled cortical impact traumatic brain injury on day 0. Means and standard errors for data from mice that completed the entire protocol were included. No significant differences were found between groups at baseline prior to TBI. After TBI, mice that did not receive antibody had impaired ability to reach the hidden platform (days 18 to 21). In contrast, mice treated with the anti-Aβ antibody m266 starting 12-16 hours before TBI reached the hidden platform with significantly shorter swim distances than placebo-treated TBI mice (p=0.001). The group treated with anti-Aβ antibody prior to TBI performed no differently from the PDAPP sham mice that never received TBI (p=0.19). Overall, none of the PDAPP groups improved over time following TBI or sham injury (p=0.46), whereas there was significant improvement over time in the PDAPP mice before TBI or sham injury (days −10 to −6, p=0.000002).

FIG. 1B shows the distance to reach the platform for wild-type (WT) mice, with PDAPP+TBI+m266 data superimposed for comparison. Prior to TBI, PDAPP mice performed worse than WT mice in hidden platform testing (p<10⁻⁶), but not during visible platform testing (p=0.25). After TBI, WT mice and antibody-treated PDAPP mice had similar swim distances, indicating that antibody treatment negated the deleterious effects of the PDAPP genotype. Sham-treated WT mice had similar performances before and after the procedure and sham-treated WT mice did not differ from naïve mice (p=0.51). This demonstrates that the sham injury had no effect on cognitive performance.

FIG. 1C shows the time to reach the platform in PDAPP mice. Antibody-treated mice reached the hidden platform with shorter swim times than placebo-treated TBI mice, although this did not reach statistical significance (p=0.136). The discrepancy between distance and time measures occurred because the placebo-treated mice swam significantly faster than the antibody-treated and sham-TBI mice (p=0.026, repeated measures ANOVA). In this situation, the distance measure is considered more indicative of cognitive performance (Crawley, 2000).

FIG. 1D shows the time to reach the platform for wild-type (WT) mice, with PDAPP+TBI+m266 antibody data superimposed for comparison.

The PDAPP mice that received anti-Aβ antibody before TBI were similar on the visible platform test (FIG. 1A, days 13-15) to those that received placebo (p=0.27). This demonstrates that the groups did not differ in their ability to swim and see the platform or in their motivation to escape from the water (Crawley, 2000). The improved performance in the mice given anti-Aβ antibody also cannot be readily accounted for by systematic differences between groups that were present prior to TBI; there were no significant differences in hidden platform performance between the PDAPP mice that were going to receive the anti-Aβ antibody and those that were not (FIG. 1A, days −10 to −6, p=0.23). This analysis included only those animals that completed the entire protocol and excluded mice that died or were disqualified due to inability to perform the visible platform portion of the water maze. There was no significant correlation between the pre-TBI and post-TBI performance of each individual mouse (R²=0.0344, data not shown). There was therefore no attempt to normalize post-TBI performance using pre-TBI performance. There were no differences in performance between male and female mice overall, (p=0.49) nor any interaction between sex and group assignment (p=0.73).

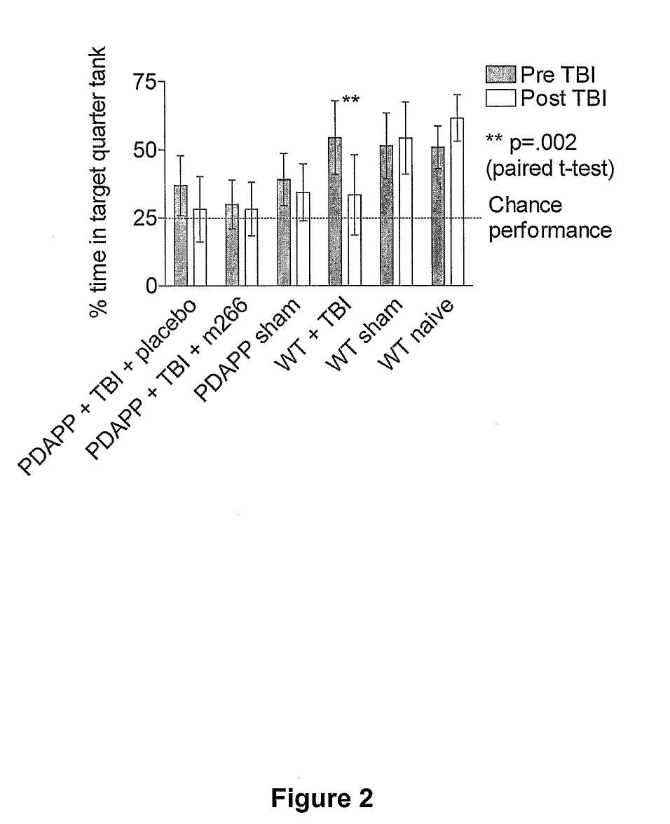

In the probe trial (FIG. 2), mice in all of the PDAPP groups and the WT TBI group appeared on average to have spent very little to no more time in the target quadrant than would be predicted by chance (25%). Error bars in FIG. 2 represent 95% confidence intervals. Similar results were obtained in an analysis of time spent in the exact area where the platform had been (not shown). As expected, the WT sham and WT naïve groups spent around half of their time in the target quadrant, demonstrating spatial memory. The probe trial is an important test for true spatial memory (Crawley, 2000). This suggests that the improvement in hidden platform performance in antibody-treated mice is due to aspects of cognitive function other than spatial memory.

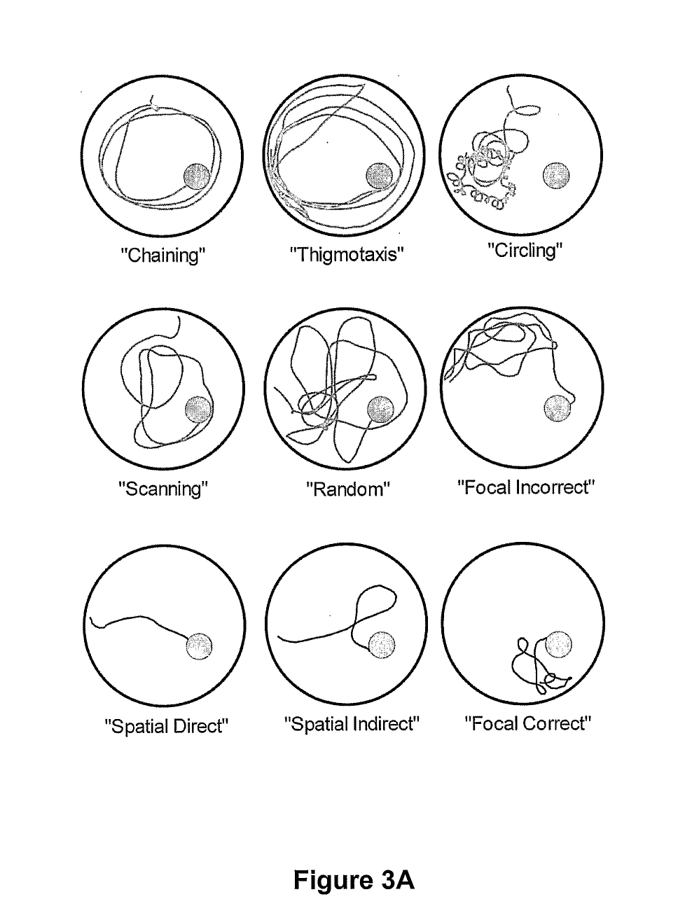

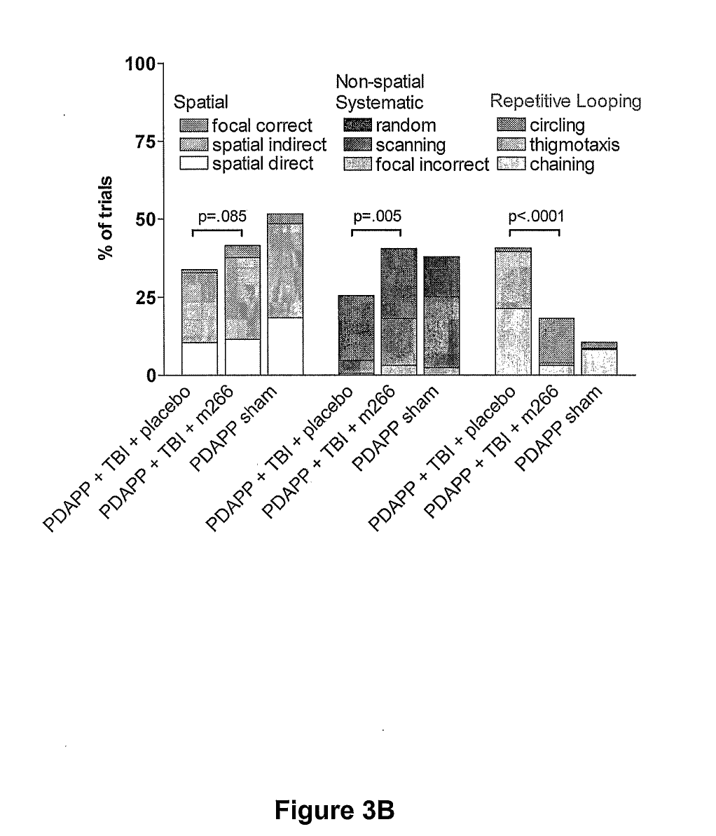

Instead, differences in search strategy appear to be involved in the improved performance of anti-Aβ antibody treated PDAPP mice (FIG. 3). A predominant search strategy (Janus, 2004) was assigned to each hidden platform trial in a blinded fashion. FIG. 3A shows examples of swim paths used to determine search strategy. Each trace represents the swim path as recorded by the computer tracking system during a single swim in the pool. The location of the hidden platform is represented by the gray circle. Top row traces are examples of repetitive looping strategies. Middle row traces represent non-spatial, systematic strategies. Bottom row traces typify spatial strategies. A few trials did not fit any of these categories.

Search strategy use was improved in antibody-treated PDAPP mice (FIG. 3B) during hidden platform testing following TBI. Antibody-treated PDAPP mice used a systematic but non-spatial search strategy during a larger proportion of the trials combined across all 4 days of hidden platform testing (107/288 trials, 37%) than placebo-treated PDAPP mice (48/192 trials, 25%. p=0.005, Chi-square). In contrast, the placebo-treated mice used strategies involving repetitive looping paths more often (78/192, 41%) than the antibody-treated mice (52/288, 18%, p<0.0001). There were no detectable differences in search strategy between groups prior to TBI (p=0.91) to suggest that the groups were unbalanced at baseline. There was marginally greater use of true spatial strategies in the antibody-treated TBI mice (120/288, 42%) than in the placebo-treated TBI mice (65/192, 34%) though this did not reach statistical significance (p=0.085). Strategy use in m266-treated mice subjected to TBI was similar to that of uninjured, sham PDAPP mice.

Anti-Aβ antibody-treated PDAPP mice swam more slowly (15.32±0.71 cm/s) than placebo-treated mice (18.14±0.87 cm/s) following TBI. This difference was statistically significant (p=0.026, repeated measures ANOVA). There were no changes in swim speed for either group across the 4 days of hidden platform training. Changes in search strategy use appear to underlie the differences in swim speed. The thigmotaxis and chaining favored by the placebo-treated mice are associated with higher swim speeds (19.31±1.1 and 19.27±0.56 cm/s respectively) than the circling (12.18±1.1 cm/s) and non-spatial, systematic strategies (16.3±0.28 m/s) used predominantly by the anti-Aβ antibody treated mice. Overall, when there is a discrepancy between distance and time measures, the distance measure may be more indicative of cognitive performance; a relatively cognitively intact mouse may swim slowly but directly to the platform, whereas a more cognitively impaired mouse may swim more quickly but less directly to the platform.

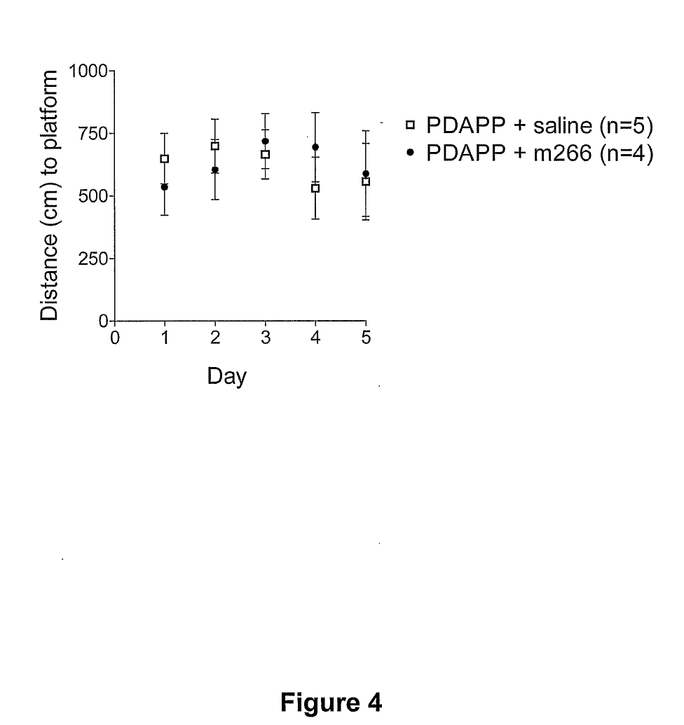

Treatment of young PDAPP mice not subjected to TBI did not benefit water maze performance (FIG. 4). In a separate experiment, 4-6 month old PDAPP mice on C57B16 background were given 500 μg/week of m266 or saline over 4 weeks in a blinded fashion. There was no effect of m266 treatment on performance in the hidden platform portion of the Morris water maze relative to saline-treatment (p=0.78, Repeated measures ANOVA). Statistical power calculations based on sample sizes of 5 and 4 were performed using Monte Carlo simulations followed by Repeated Measures ANOVA; if there had been an effect of m266 treatment the same size as was seen in the mice subjected to TBI, it would have been detected with a p-value ranging between 0.018 to 0.038. This indicates that there was no effect of m266 treatment on PDAPP mice not subjected to TBI. This suggests that m266 does not generally boost cognitive function in young PDAPP mice, prior to the development of Aβ deposition. Instead, it indicates that anti-Aβ antibody treatment specifically attenuated the adverse effects on water maze performance due to TBI.

Histological Effects of TBI and Anti-Aβ Antibody Treatment

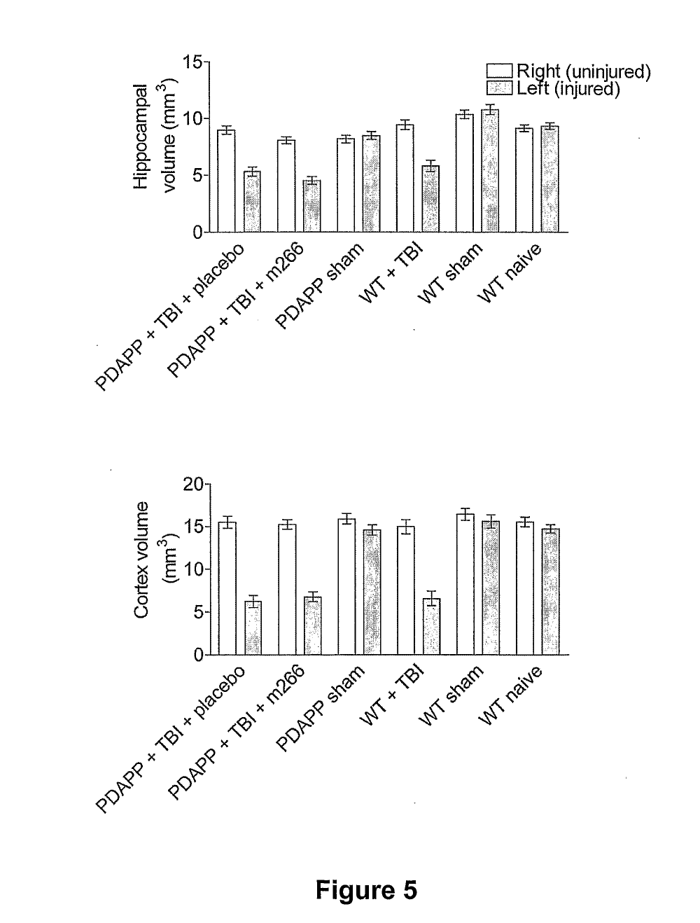

Histologically, there were no significant differences in the overall size of the lesions between the brain-injured PDAPP mice that received anti-Aβ antibody pretreatment and those that received placebo (FIG. 5). There were no gross anatomical differences between antibody treated and untreated PDAPP TBI mice and no visible lesions after sham treatment. No differences were found in volumes ipsilateral or contralateral to the TBI lesion in the antibody treated versus placebo-treated PDAPP+TBI groups. There was no effect of sham treatment on cortical or hippocampal volumes. PDAPP mice as a group had slightly but significantly smaller hippocampi both ipsilateral (left) and contralateral (right) to TBI compared with WT mice (p=0.002) but there was no difference in the ratio of injured to uninjured hippocampal volumes (p=0.97) or cortical volumes (p=0.49) to suggest a differential structural susceptibility to trauma in PDAPP mice.

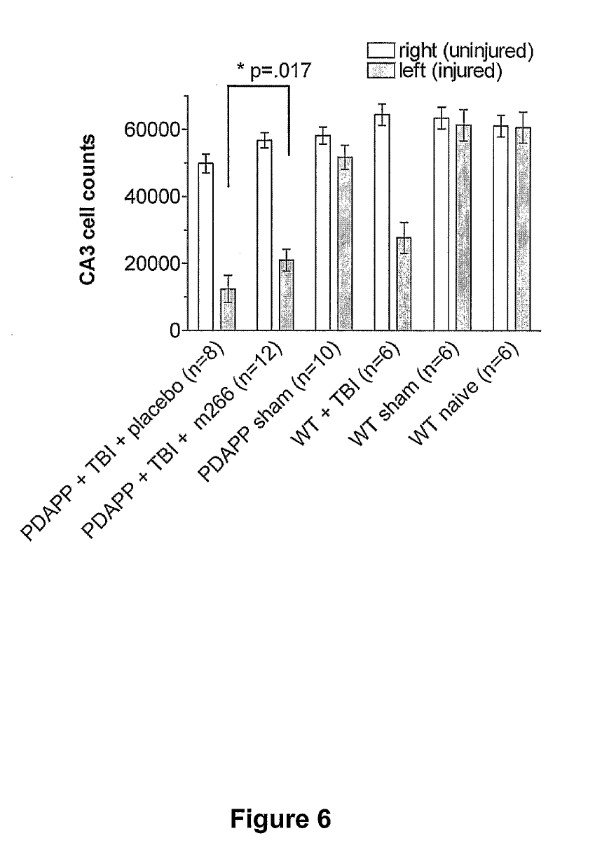

However, there was a small but significant reduction in ipsilateral CA3 neuronal loss in the antibody treated PDAPP mice compared to those given placebo (FIG. 6). The ipsilateral CA3 region has been shown to be especially vulnerable to controlled cortical impact TBI in mice (Smith et al., 1995). No mice had substantial contralateral CA3 cell loss, and none of the sham-treated mice had detectable CA3 cell loss compared to naïve mice. The CA1 region and dentate gyrus (DG), while damaged, did not show the same degree of cell loss as CA3. PDAPP mice subjected to TBI had a 74% loss of ipsilateral CA3 neurons as compared to mice subjected to sham TBI. With anti-Aβ treatment, CA3 cell loss was reduced to 59% (p=0.017, Mann-Whitney U test). This was similar to the 55% cell loss seen in WT mice subjected to TBI. The effects of TBI on contralateral CA3 counts in PDAPP mice was not statistically significant (p=0.12). There were no differences between sham treated and naïve WT mice in either ipsilateral or contralateral CA3 counts.

Most PDAPP mice (80%) showed no evidence of any Aβ or amyloid deposition. Similar results have been obtained in comparably aged PDAPP+/−mice not subjected to TBI (Johnson-Wood et al., 1997). The remaining 20% of the mice showed rare, very small cortical and white matter Aβ plaques, some of which were also thioflavine-S positive indicating the presence of true, β-sheet containing amyloid. These mice were evenly distributed between the sham, placebo-treated and injured, and the antibody-treated injured PDAPP groups. Overall, 6 of 30 PDAPP mice, 2 from each of the three PDAPP groups, had minimal but detectable Aβ deposition and 24 had no deposition. A 15 month-old PDAPP mouse never subjected to TBI was used as a positive control for the detection of Aβ deposition and thio-S positive amyloid. Sections from this animal consistently revealed Aβ deposition and amyloid plaques. A 5-6 month-old WT mouse subjected to TBI was used as a negative control. There was no evidence of Aβ deposition or amyloid in sections from this animal. Thus, there was unlikely to have been a major acceleration of Aβ deposition or amyloid formation caused by the TBI at this time point.

Increased BrdU-NeuN Double Labeled Cells Following TBI and Anti-Aβ Antibody Treatment

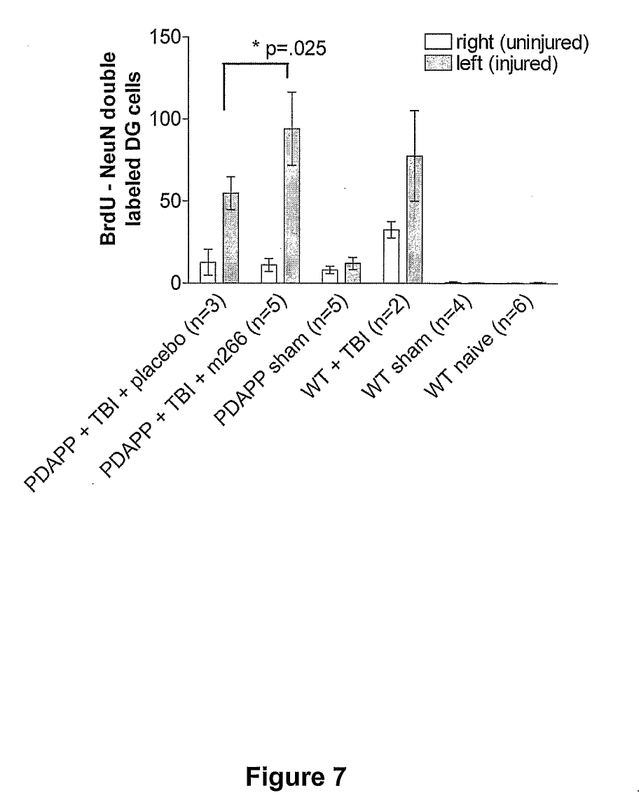

TBI increased the number of BrdU-NeuN double labeled cells in the dentate gyrus, and anti-Aβ antibody treatment further increased the numbers of these cells (FIG. 7). Traumatic brain injury induced a large increase in the number of double labeled cells in both PDAPP and WT mice (p=0.025, Mann-Whitney U test). This has been previously reported for wild-type mice and rats (Dash et al., 2001; Kernie et al., 2001; Chen et al., 2003). There were more double labeled cells in mice treated with anti-Aβ antibody than in placebo treated mice (p=0.025, Mann-Whitney U test). There were very few double labeled cells in sham treated WT mice, whereas sham-treated PDAPP mice had increased numbers (p<0.02). Contralateral dentate gyms counts of BrdU, NeuN-positive double labeled cells were no different between groups of PDAPP mice (p>0.05). Also, we found that sham-treated PDAPP mice had higher basal levels of double labeled cells than sham-treated wild type mice (p<0.02). Similar results have been obtained in some (Jin et al., 2004) but not all lines of APP transgenic mice (Haughey et al., 2002).

NeuN is a neuronal marker, and BrdU incorporates into the DNA of newly dividing cells. Thus, there was preliminary evidence for an increase in the generation of new neurons after TBI in antibody treated PDAPP mice. Several interpretations, however, are possible as the exact nature of these double labeled cells has been the subject of considerable controversy (Rakic, 2002).

Systemic administration of the anti-Aβ antibody m266 significantly reduced Morris water maze deficits in young PDAPP mice subjected to moderate TBI. The main underlying cognitive mechanism appeared to be improvements in search strategy selection rather than in true spatial memory. There were accompanying significant decreases in hippocampal CA3 cell loss and apparent increases in newly generated dentate gyms neurons in the antibody treated mice. However, there were no differences between the antibody treated and untreated group in the gross size of the lesions in hippocampus and cortex, and minimal to no deposition of Aβ and amyloid formation in any of the groups. Thus, the overall histological effects of the anti-Aβ antibody treatment were relatively subtle compared with the magnitude of the cognitive benefit. Taken together, these results suggest that m266 treatment at least partially dissociated the physical lesion from the cognitive sequelae of TBI.

Acute changes in soluble Aβ handling and metabolism after TBI likely contributes to cognitive impairment. As there was no significant deposition of insoluble Aβ in these young mice, it is likely that antibody treatment blunts effects of the rapid rise in soluble Aβ levels seen transiently after TBI (Smith et al., 1998). In the setting of TBI, the blood brain barrier is disrupted (Smith et al., 1995) and anti-Aβ antibodies are likely to have direct access to Aβ in the brain, in addition any effects on Aβ efflux from brain to blood (DeMattos et al., 2001).

While the discovery has been described in terms of various specific embodiments, those skilled in the art will recognize that the discovery can be practiced with modification of these embodiments within the spirit and scope of the claims.

### REFERENCES

Arvidsson A, Kokaia Z Lindvall O (2001) N-methyl-D-aspartate receptor-mediated increase of neurogenesis in adult rat dentate gyrus following stroke. Eur J Neurosci 14:10-18.
Bloom B S, de Pouvourville N, Straus W L (2003) Cost of illness of Alzheimer's disease: how useful are current estimates? Gerontologist 43:158-164.
Braak H. Braak E (1997) Frequency of stages of Alzheimer-related lesions in different age categories. Neurobiol Aging 18:351-357.
Chen G, Chen K S, Knox J, Inglis J, Bernard A, Martin S J, Justice A, McConlogue L, Games D, Freedman S B, Morris R G (2000) A learning deficit related to age and beta-amyloid plaques in a mouse model of Alzheimer's disease. Nature 408:975-979.
Chen X H, Iwata A, Nonaka M, Browne K D, Smith D H (2003) Neurogenesis and glial proliferation persist for at least one year in the subventricular zone following brain trauma in rats. Journal of Neurotrauma 20:623-631.
Chen X H, Siman R, Iwata A, Meaney D F, Troianowski J Q, Smith D H (2004) Long-term accumulation of amyloid-beta, beta-secretase, presenilin-1, and caspase-3 in damaged axons following brain trauma. Am J Pathol 165:357-371.
Crawley J N (2000) Whats wrong with my mouse? Chapter 6 Learning and Memory. New York: Wiley-Liss.
D'Hooge R, De Deyn P P (2001) Applications of the Morris water maze in the study of learning and memory. Brain Research—Brain Research Reviews 36:60-90.
Dash P K, Mach S A, Moore A N (2001) Enhanced neurogenesis in the rodent hippocampus following traumatic brain injury. Journal of Neuroscience Research 63:313-319.
Davies L, Wolska B, Hilbich C, Multhaup G, Martins R, Simms G, Beyreuther K, Masters C L (1988) A4 amyloid protein deposition and the diagnosis of Alzheimer's disease: prevalence in aged brains determined by immunocytochemistry compared with conventional neuropathologic techniques. Neurology 38:1688-1693.
DeMattos R B, Bales K R, Cummins D J, Dodart J C, Paul S M, Holtzman D M (2001) Peripheral anti-A beta antibody alters CNS and plasma A beta clearance and decreases brain A beta burden in a mouse model of Alzheimer's disease. Proc Natl Acad Sci USA 98:8850-8855.
Dixon C E, Clifton G L, Lighthall J W, Yaghmai A A, Hayes R L (1991) A controlled cortical impact model of traumatic brain injury in the rat. Journal of Neuroscience Methods 39:253-262.
Dodart J C, Bales K R, Gannon K S, Greene S J, DeMattos R B, Mathis C, DeLong C A, Wu S, Wu X, Holtzman D M, Paul S M (2002) Immunization reverses memory deficits without reducing brain Abeta burden in Alzheimer's disease model. Nat Neurosci 5:452-457.
Games D, Adams D, Alessandrini R, Barbour R, Berthelette P, Blackwell C, Carr T, Clemens J, Donaldson T, Gillespie F, et al. (1995) Alzheimer-type neuropathology in transgenic mice overexpressing V717F beta-amyloid precursor protein. Nature 373:523-527.
Graham D I, Gentleman S M, Lynch A, Roberts G W (1995) Distribution of beta-amyloid protein in the brain following severe head injury. Neuropathology & Applied Neurobiology 21:27-34.
Hartman R E, Laurer H, Longhi L, Bales K R, Paul S M, McIntosh T K, Holtzman D M (2002) Apolipoprotein E4 influences amyloid deposition but not cell loss after traumatic brain injury in a mouse model of Alzheimer's disease. J Neurosci 22:10083-10087.
Haugley N J, Nath A, Chan S L, Borchard A C, Rao M S, Mattson M P (2002) Disruption of neurogenesis by amyloid beta-peptide, and perturbed neural progenitor cell homeostasis, in models of Alzheimer's disease. J Neurochem 83:1509-1524.
Hebert L E, Scherr P A, Bienias J L, Bennett D A, Evans D A (2003) Alzheimer disease in the US population: prevalence estimates using the 2000 census. Archives of Neurology 60:1119-1122.
Holtzman D M, Bales K R, Tenkova T, Fagan A M, Parsadanian M, Sartorius L J, Mackey B, Olney J, McKeel D, Wozniak D, Paul S M (2000) Apolipoprotein E isoform-dependent amyloid deposition and neuritic degeneration in a mouse model of Alzheimer's disease. Proc Natl Acad Sci USA 97:2892-2897.
Hsiao K, Chapman P, Nilsen S, Eckman C, Harigaya Y, Youkin S, Yang F. Cole G (1996) Correlative memory deficits, Aβ elevation, and amyloid plaques in transgenic mice. Science 274:99-102.
Huber A, Gabbert K, Kelemen J, Cervos-Navarro J (1993) Density of amyloid plaques in brains after head trauma. 2nd International Neurotrauma Symposium abstracts: P108.
Huitron-Resendiz S, Sanchez-Alavez M, Gallegos R, Berg G, Crawford E, Giacchino J L, Games D, Henriksen S J, Criado J R (2002) Age-independent and age-related deficits in visuospatial learning, sleep-wake states, thermoregulation and motor activity in PDAPP mice. Brain Res 928:126-137.
Ikonomovic M D, Uryu K, Abrahamson E E, Ciallella J R, Trojanowski J Q, Lee V M, Clark R S, Marion D W Wisniewski S R, DeKosky S T (2004) Alzheimer's pathology in human temporal cortex surgically excised after severe brain injury. Exp Neurol 190:192-203
Janus C (2004) Search strategies used by APP transgenic mice during navigation in the Morris water maze. Learning & Memory 11:337-346.
Janus C, Pearson J, McLaurin J, Mathews P M, Jiang Y, Schmidt S D, Chishti M A, Horne P, Heslin D, French J, Mount H T, Nixon R A, Mercken M, Bergeron C, Fraser P E, St George-Hyslop P, Westaway D (2000) A beta peptide immunization reduces behavioural impairment and plaques in a model of Alzheimer's disease. Nature 408:979-982.
Jin K, Galvan V, Xie L, Mao X O, Gorostiza O F, Bredesen D E, Greenberg D A (2004) Enhanced neurogenesis in Alzheimer's disease transgenic (PDGF-APPSw,Ind)mice. Proc Natl Acad Sci USA 101:13363-13367.
Johnson-Wood K, Lee M, Motter R, Hu K, Gordon G, Barbour R, Khan K, Gordon M, Tan H, Games D, Lieberburg I, Schenk D, Seubert P, McConlogue L (1997) Amyloid precursor protein processing and A beta42 deposition in a transgenic mouse model of Alzheimer disease. Proc Natl Acad Sci USA 94:1550-1555.
Kernie S G, Erwin T M, Parada L F (2001) Brain remodeling due to neuronal and astrocytic proliferation after controlled cortical injury in mice. Journal of Neuroscience Research 66:317-326.
Kuhn H G, Dickinson-Anson H, Gage F H (1996) Neurogenesis in the dentate gyrus of the adult rat: age-related decrease of neuronal progenitor proliferation. J Neurosci 16:2027-2033.
Morgan D, Diamond D M, Gottschall P E, Ugen K E, Dickey C, Hardy J. Duff K, Jantzen P, DiCarlo G, Wilcock D, Connor K, Hatcher J Hope C, Gordon M, Arendash G W (2000) A beta peptide vaccination prevents memory loss in an animal model of Alzheimer's disease. Nature 408:982-985.
Morris R (1984) Developments of a water-maze procedure for studying spatial learning in the rat. Journal of Neuroscience Methods 11:47-60.
Morris R G, Garrud P, Rawlins J N, O'Keefe J (1982) Place navigation impaired in rats with hippocampal lesions. Nature 297:681-683.
Mortimer J A, van Duijn C M, Chandra V, Fratiglioni L, Graves A B, Heyman A, Jorm A F, Kokmen E, Kondo K, Rocca W A (1991) Head trauma as a risk factor for Alzheimer's disease: a collaborative re-analysis of case-control studies. EURODEM Risk Factors Research Group. International Journal of Epidemiology 20 Suppl 2:S28-35.
Murai H, Pierce J E, Raghupathi R, Smith D H, Saatman K E, Trojanowski J Q, Lee V M, Loring J F, Eckman C, Younkin S, McIntosh T K (1998) Twofold overexpression of human beta-amyloid precursor proteins in transgenic mice does not affect the neuromotor, cognitive, or neurodegenerative sequelae following experimental brain injury. J Comp Neurol 392:428-438.
Nakagawa Y, Nakamura M, McIntosh T K, Rodriguez A, Berlin J A, Smith D H, Saatman K E, Raghupathi R, Clemens J, Saido T C, Schmidt M L, Lee V M, Trojanowski J Q (1999) Traumatic brain injury in young, amyloid-beta peptide overexpressing transgenic mice induces marked ipsilateral hippocampal atrophy and diminished Abeta deposition during aging. J Comp Neurol 411:390-398.
Narayan R K, Michel M E, Ansell B, Baethmann A, Biegon A, Bracken M B, Bullock M R, Choi S C, Clifton G L, Contant C F, Coplin W M, Dietrich W D, Ghajar J, Grady S M, Grossman R G, Hall E D, Heetderks W, Hovda D A, Jallo J, Katz R L, Knoller N, Kochanek P M, Maas A I, Majde J, Marion D W, Marmarou A, Marshall L F, McIntosh T K, Miller E, Mohberg N, Muizelaar J P, Pitts L H, Quinn P, Riesenfeld G, Robertson C S, Strauss K I, Teasdale G, Teinkin N, Tuma R, Wade C, Walker M D, Weinrich M, Whyte J, Wilberger J, Young A B, Yurkewicz L (2002) Clinical trials in head injury. Journal of Neurotrauma 19:503-557.
Plassman B L, Havlik R J, Steffens D C, Helms M J, Newman T N, Drosdick D, Phillips C, Gau B A, Welsh-Bohmer K A, Burke J R, Guralnik J M, Breitner J C (2000) Documented head injury in early adulthood and risk of Alzheimer's disease and other dementias. Neurology 55:1158-1166.
Rakic P (2002) Adult neurogenesis in mammals: an identity crisis. J Neurosci 22:614-618.
Roberts G W, Gentleman S M, Lynch A, Graham D I (1991) beta A4 amyloid protein deposition in brain after head trauma. Lancet 338:1422-1423.
Roberts G W Gentleman S M, Lynch A, Murray L, Landon M, Graham D I (1994) Beta amyloid protein deposition in the brain after severe head injury: implications for the pathogenesis of Alzheimer's disease. J Neurol Neurosurg Psychiatry 57:419-425.
Smith D H, Chen X H, Iwata A, Graham D I (2003) Amyloid beta accumulation in axons after traumatic brain injury in humans. Journal of Neurosurgery 98:1072-1077.
Smith D H, Soares H D, Pierce J S, Perlman K G, Saatman K E, Meaney D F, Dixon C E, McIntosh T K (1995) A model of parasagittal controlled cortical impact in the mouse: cognitive and histopathologic effects. J Neurotrauma 12:169-178.
Smith D H, Chen X H, Nonaka M, Trojanowski J Q, Lee V M, Saatman K E, Leoni M J, Xu B N, Wolf J A, Meaney D F (1999) Accumulation of amyloid beta and tau and the formation of neurofilament inclusions following diffuse brain injury in the pig. J Neuropathol Exp Neurol 58:982-992.
Smith D H, Nakamura M, McIntosh T K, Wang J. Rodriguez A, Chen X H, Raghupathi R, Saatman K E, Clemens J, Schmidt M L, Lee V M, Trojanowski J Q (1998) Brain trauma induces massive hippocampal neuron death linked to a surge in beta-amyloid levels in mice overexpressing mutant amyloid precursor protein. Am J Pathol 153:1005-1010.
Thurman D J, Alverson C, Dunn K A, Guerrero J, Sniezek J E (1999) Traumatic brain injury in the United States: A public health perspective. Journal of Head Trauma Rehabilitation 14:602-615.
Uryu K, Laurer H, McIntosh T, Pratico D, Martinez D, Leight S, Lee V M, Trojanowski J Q (2002) Repetitive mild brain trauma accelerates Abeta deposition, lipid peroxidation, and cognitive impairment in a transgenic mouse model of Alzheimer amyloidosis. J Neurosci 22:446-454.

1. A method of treating traumatic brain injury (TBI) in a patient which comprises administering an effective amount of an anti-Aβ antibody to that patient.

2. A method of preventing, attenuating, reversing, or improving at least one symptom or sign of TBI in a patient comprising administering an effective amount of an anti-Aβ antibody to that patient.

3. The method of claim 2 wherein a sign or symptom of TBI includes impaired cognitive function, altered behavior, emotional dysregulation, seizures, headaches, impaired nervous system structure or function, and an increased risk of development of Alzheimer's disease.

4. The method of claim 1 wherein the antibody comprises an antibody that therapeutically attenuates the toxic effects of the Aβ peptide in a living mammal.

5. The method of claim 1 wherein the patient is a human.

6. The method of claim 1 wherein the anti-Aβ antibody binds an epitope within the region between amino acids 13 and 28 of an Aβ peptide.

7. The method of claim 6 wherein the anti-Aβ antibody is an antibody that binds the same epitope of an antibody comprising a light chain comprising the amino acid sequence of SEQ ID NO:11 and a heavy chain comprising the amino acid sequence of SEQ ID NO:12.

8. The method of claim 6 wherein the anti-Aβ antibody is an antibody comprising a light chain comprising the amino acid sequence of SEQ ID NO:11 and a heavy chain comprising the amino acid sequence of SEQ ID NO:12.

9. The method of claim 1 wherein the administration comprises an effective systemic route of administration.

10. The method of claim 1 wherein the administration comprises an effective local route of administration including directly within the central nervous system.

11. A medicinal composition useful to treat TBI comprising a medicinally effective amount of an anti-Aβ antibody adapted for administration to a living human patient suffering from TBI along with a pharmaceutically acceptable excipient or excipients.

12. A medicinal kit useful to treat TBI comprising a container containing a medicinally effective amount of an anti-Aβ antibody adapted for systemic administration to a living human patient suffering from TBI, a pharmaceutically acceptable excipient or excipients, and any medical devices to be used for said administration.

13. The use of an effective amount of an anti-Aβ antibody for the manufacture of a medicament to treat TBI in a patient.

14. The use of an effective amount of an anti-Aβ antibody for the manufacture of a medicament to prevent, attenuate, reverse, or improve at least one symptom or sign of TBI in a patient.

15. The use of claim 14 wherein the wherein a sign or symptom of TBI includes impaired cognitive function, altered behavior, emotional dysregulation, seizures, headaches, impaired nervous system structure or function, and an increased risk of development of Alzheimer's disease.

16. The use of claim 13 wherein the anti-Aβ antibody binds an epitope within the region between amino acids 13 and 28 of an Aβ peptide.

17. The use of claim 16 wherein the anti-Aβ antibody is an antibody that binds the same epitope of an antibody comprising a light chain comprising the amino acid sequence of SEQ ID NO:11 and a heavy chain comprising the amino acid sequence of SEQ ID NO:12.

18. The use of claim 16 wherein the anti-Aβ antibody is an antibody comprising a light chain comprising the amino acid sequence of SEQ ID NO:11 and a heavy chain comprising the amino acid sequence of SEQ ID NO:12.

19. The method of claim 1 wherein the anti-Aβ antibody binds to an Aβ peptide circulating in the patient's blood and alters the Aβ peptide into soluble forms of Aβ in the patient's central nervous system and plasma.

20. The method of claim 1 wherein the anti-Aβ antibody is administered by intraperitoneal administration.


SEQUENCE LISTING (16 sequences)

SEQ ID NO: 1 - protein, 16 aa, Artificial Sequence
  Description of Artificial Sequence Synthetic peptide
RSSQSLIYSDGNAYLH

SEQ ID NO: 2 - protein, 7 aa, Artificial Sequence
  Description of Artificial Sequence Synthetic peptide
KVSNRFS

SEQ ID NO: 3 - protein, 9 aa, Artificial Sequence
  Description of Artificial Sequence Synthetic peptide
SQSTHVPWT

SEQ ID NO: 4 - protein, 5 aa, Artificial Sequence
  Description of Artificial Sequence Synthetic peptide
RYSMS

SEQ ID NO: 5 - protein, 17 aa, Artificial Sequence
  Description of Artificial Sequence Synthetic peptide
QINSVGNSTYYPDTVKG

SEQ ID NO: 6 - protein, 3 aa, Artificial Sequence
  Description of Artificial Sequence Synthetic peptide
GDY

SEQ ID NO: 7 - protein, 113 aa, Artificial Sequence
  Description of Artificial Sequence Synthetic polypeptide
DXVMTQXPLSLPVXXGQPASISCRSSQSLXYSDGNAYLHWFLQKPGQSPXLLIYKVSNRFSGVPDRFSGSGSGTDFTLKISRVEAEDXGVYYCSQSTHVPWTFGXGTXXEIKR

SEQ ID NO: 8 - protein, 112 aa, Artificial Sequence
  Description of Artificial Sequence Synthetic polypeptide
XVQLVEXGGGLVQPGGSLRLSCAASGFTFSRYSMSWVRQAPGKGLXLVAQINSVGNSTYYPDXVKGRFTISRDNXXNTLYLQMNSLRAXDTAVYYCASGDYWGQGTXVTVSS

SEQ ID NO: 9 - protein, 113 aa, Artificial Sequence
  Description of Artificial Sequence Synthetic polypeptide
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKR

SEQ ID NO: 10 - protein, 112 aa, Artificial Sequence
  Description of Artificial Sequence Synthetic polypeptide
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSS

SEQ ID NO: 11 - protein, 219 aa, Artificial Sequence
  Description of Artificial Sequence Synthetic polypeptide
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 442 aa, Artificial Sequence
  Description of Artificial Sequence Synthetic polypeptide
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 17 aa, Artificial Sequence
  Description of Artificial Sequence Synthetic peptide
QINSVGXXXYYPDTVKG

SEQ ID NO: 14 - protein, 112 aa, Artificial Sequence
  Description of Artificial Sequence Synthetic polypeptide
XVQLVEXGGGLVQPGGSLRLSCAASGFTFSRYSMSWVRQAPGKGLXLVAQINSVGXXXYYPDXVKGRFTISRDNXXNTLYLQMNSLRAXDTAVYYCASGDYWGQGTXVTVSS

SEQ ID NO: 15 - protein, 112 aa, Artificial Sequence
  Description of Artificial Sequence Synthetic polypeptide
EVQLVESGGGLVQPGGSLRLSCAASGFTFSRYSMSWVRQAPGKGLELVAQINSVGXXXYYPDTVKGRFTISRDNAKNTLYLQMNSLRAEDTAVYYCASGDYWGQGTLVTVSS

SEQ ID NO: 16 - protein, 442 aa, Artificial Sequence
  Description of Artificial Sequence Synthetic polypeptide
EVQLVESGGGLVQPGGSLRLSCAASGFTFSRYSMSWVRQAPGKGLELVAQINSVGXXXYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK